(19) United States
(12) Patent Application Publication
(10) Pub. No.: US 2012/0083772 A1
(43) Pub. Date: Apr. 5, 2012

(54) CORNEAL TREATMENT SYSTEM AND METHOD

(75) Inventors: Roy Scott Rubinfeld, Bethesda, MD (US); Sandy T. Feldman, Del Mar, CA (US); Raymond A. Hartman, Carlsbad, CA (US)

(73) Assignee: CURVERIGHT LLC, Lewes, DE (US)

(21) Appl. No.: 13/034,488

(22) Filed: Feb. 24, 2011

Related U.S. Application Data
(60) Provisional application No. 61/388,362, filed on Sep. 30, 2010.

(51) Int. Cl. A61F 9/007; A61P 27/02; A61K 49/00; A61K 33/18; A61K 38/44

(52) U.S. Cl. 606/4; 424/671; 424/94.4; 424/9.6

(57) ABSTRACT

A system for bilateral or monocular photochemical cross-linking of corneal collagen employs selectable light in a selected wavelength band as the excitation source and riboflavin as the photosensitizer. The system has an illumination source which may have multi-spectral capability, light guides for delivery of light to the optical head for projection onto the corneal surface, selectable radiation patterns to accommodate individual corneal architecture, and red light phototherapy to limit apoptosis and accelerate healing time. Aiming beams provide alignment of the optical head to the patient cornea. A microprocessor-controlled rotary solenoid mechanical shutter provides discontinuous illumination for tissue reoxygenation, and devices and methods may be included for the in situ determination of oxygen utilization and the riboflavin content of the cornea.

### RELATED APPLICATION

The present application claims the benefit of co-pending U.S. provisional pat. App. Ser. No. 61/388,362, filed Sep. 30, 2010, the contents of which are incorporated herein by reference in their entirety.

### BACKGROUND

1. Field of the Invention

The present invention relates generally to ophthalmic surgery and treatment, and is particularly concerned with a photochemical treatment system and method for strengthening the cornea when weakened by various conditions.

2. Related Art

The cornea, the anterior structure of the eye, is a convex transparent barrier that serves to maintain the intact structure of the eye and focus light onto the retina. The cornea derives its structural strength, shape and integrity from corneal collagen. The strength of the intertwined collagen strands is a function of covalent cross-links established between and within collagen strands and between collagen and glycoproteins in the matrix. In structurally robust corneas, an enzyme called lysyl oxidase performs the collagen cross-linking function in a process called oxidative deamination using molecular oxygen present in the tissue. The biomechanical strength of corneal collagen can be reduced by a number of conditions including iatrogenic effect from surgical intervention, prosthesis, or medications, or the cause of corneal weakness can be congenital, idiopathic or due to microbial causes or trauma. In these cases of corneal weakness, interventional strategies to strengthen the collagen are often employed.

Strengthening the weakened corneal collagen has been attempted by chemical, physical and photochemical means. Chemical means such as the introduction into the cornea of glutaraldehyde, formaldehyde, glyceraldehyde, ribose, glucose and beta-nitro aliphatic alcohols have been investigated. Most of these chemical cross-linking methods have been abandoned due to concerns with toxicity and efficacy. Physical methods of strengthening corneal collagen include dehydrothermal treatment (desiccation of water from collagen), thermal heating and UVC or gamma radiation. Many of these techniques have drawbacks including denaturization and degradation of collagen, damaging or killing keratocytes, and potentially toxic side effects.

The most promising clinical method of cross-linking compromised corneal collagen is photochemical cross-linking, which has been in human clinical use for the treatment of keratoconus and ectasia for some time. This method of photochemical cross-linking uses a photosensitizer, usually riboflavin monophosphate, and UVA light to create singlet oxygen in stromal collagen. It is believed that the singlet oxygen reacts to convert lysine side-chains of collagen fibrils to allysine residues that spontaneously condense to cross link the collagen fibrils. Essentially this mimics the reaction caused by the natural enzyme lysyl oxidase. Studies have been done to show that singlet oxygen is the cross-linking agent which predominates over other potential reactive oxygen species. Photochemical cross-linking of the cornea has been demonstrated to stop and reverse the progression of compromised collagen in keratoconus and ectasia and thousands of patients have received this treatment with few serious adverse events.

Photochemical treatment, sometimes called photodynamic therapy, is comprised of three elements 1) excitation light, 2) photosensitizer molecules and 3) molecular oxygen. The goal of the chemical reaction is to create singlet oxygen and the amounts of each of the three variables of the reaction (light, photosensitizer, oxygen) determine the rate and amount of singlet oxygen produced. During the reaction, the molecular oxygen in the tissue is depleted. Available molecular oxygen in the tissue has often been the most limiting aspect of photochemical and photodynamic therapy. When tissue oxygen content is too low, the photochemical reaction produces little singlet oxygen. Instead, the reaction converts water into hydrogen peroxide that is cytotoxic and can stimulate the wound healing response with negative consequences. When the tissue oxygen content is relatively high, the photochemical reaction produces singlet oxygen at a rate approaching 100:1 singlet oxygen over hydrogen peroxide. Keeping the molecular oxygen levels high is a critical aspect of photochemical cross-linking.

The dynamics of the photochemical reaction depend upon the wavelength and intensity of the excitation light, the concentration of the photosensitizer and the concentration of oxygen. The only controls available on current commercial cross-linking devices are a variable intensity control for the emission and selection of the radiation spot size. In most prior art systems, a circular spot of 8-10 mm of excitation light is used on the cornea. The spot size is generally selected to protect the limbal and peripheral areas of the eye from the UV radiation.

The wavelength of the excitation light determines the absorption characteristics of the photosensitizer, and for riboflavin the maximum absorbance occurs in the UV light spectrum at around 365 nm and 445 nm. Since the absorption of the light follows the Beer-Lambert law, excitation light at these wavelengths is absorbed quickly and penetrates to the least depth into the tissue. The normal cornea is nominally around 500 microns in depth, and a layer of endothelial cells defines the back of the cornea. The depth of penetration of the UVA light into the cornea is a function of the absorbance of the photosensitizer at various wavelengths and the concentration and distribution of photosensitizer molecules. This depth of penetration is a critical value in corneal cross-linking because too little penetration gives shallow, perhaps insufficient cross-linking, and too much penetration may put the endothelium at risk.

The dynamics of the procedure for light penetration and the conversion of molecular oxygen to singlet oxygen depend upon the concentration of the photosensitizer in the tissue and the concentration of oxygen in the tissue. None of the currently marketed devices for photochemical cross-linking provides information on these two key variables to the surgeon.

There are several cautions to be observed when using UVA light and riboflavin for corneal cross-linking. The first caution is that UVA light can have cytotoxic effects on all living cells, and in particular, the corneal endothelial cells on the posterior layer of the cornea can be destroyed by excess UVA light or reactive oxygen species (ROS). It is believed that endothelial cells have limited regenerative capability and significant damage to the endothelium may be irreversible. The endothelium is a single layer of cells that governs fluid and solute transport across the posterior surface of the cornea and actively maintains the cornea in the slightly dehydrated state that is required for optical transparency. Another caution of UVA light and riboflavin photochemical treatment is that activated riboflavin can produce cytotoxic hydrogen peroxide if oxygen molecules are not available. This cytotoxic hydrogen peroxide can kill or disable healthy cells. The hydrogen peroxide also acts as a potent chemical messenger to other cells to initiate wound healing responses. These wound healing responses in the cornea may lead to edema, inflammation and differentiation of keratocytes into myofibroblasts with the production of types of collagen not conducive to optical transparency. Corneal haze and scarring can result from myofibroblast formation.

In some current standard systems, photochemical treatment of the cornea is typically conducted as follows. After topical local anesthesia of the eye, mechanical removal of the epithelium is performed to produce better riboflavin diffusion into the underlying stroma. A 0.1% riboflavin solution in 20% dextran is applied manually to the cornea every 2-5 minutes, starting 5 to 30 minutes before UVA exposure to allow stromal saturation. A UVA source at a wavelength of 365-370 nm is used to deliver an irradiance of 3 mW/cm² (a total dose of 5.4 J/cm²) to the cornea for 30 minutes. The size of the UVA spot ranges from 8-10 mm diameter. Repeated applications of riboflavin and topical anesthesia to the cornea are performed every 5 minutes

Corneal deepithelialization is performed to promote riboflavin infusion into the stroma of the eye. The deepithelialization procedure is intended to ensure that sufficient riboflavin is introduced into the eye to prevent UVA radiation in excess of 0.35 mw/cm² from reaching the endothelium. In this case, the riboflavin concentration acts as a UVA sunscreen for the deeper endothelium. There are recognized limitations and side effects to the current procedure. A significant drawback to the current devices for cross-linking is that concentration of riboflavin in the cornea is unknown. Other limitations include a limited depth of cross-linking of approximately 200 microns-250 microns, so that only the anterior third or half of the stroma is generally cross-linked. In some cases, treatment does not result in observable improvement, or does not slow or prevent disease progression. Additional adverse events may include postoperative infection/ulcer and stromal haze.

Some patients report significant discomfort, pain and worse vision lasting roughly a week, and significantly worse vision than prior to the cross-linking procedure lasting for several months. Most of the adverse side effects are a result of the surgical removal of the corneal epithelium prior to the introduction of the riboflavin. The ability to predict the clinical outcome, i.e. the improvement of a patient's best-corrected visual acuity (BCVA) or prevention of reduction of BCVA over the long term, is not high.

Commercial devices for providing the UVA irradiation are primarily limited to monocular treatment despite the fact that most patients require both eyes be treated. A bilateral device is disclosed in US2010/0057060 but this design offers very limited procedural access for the surgeon. Prior art commercial devices rely upon monochromatic UVA from LEDs or expensive lasers as the sole light source. The illumination spots of such light sources suffer from “hot spots”. LED systems are also very sensitive to thermal effects and difficult to stabilize.

Therefore, there is a need for an improved system and method for corneal treatment and strengthening.

### SUMMARY

Embodiments described herein provide for a corneal treatment system and method.

According to one embodiment, a corneal treatment system is provided which comprises a UVA light source unit for delivery of UVA light in a predetermined wavelength range, a flexible elongate light guide having a first end connected to the output of the UVA light source unit and a second end, and an optical treatment head adjustably mounted on a vertical stand and connected to the second end of the light guide, the optical head including a projection optic or lens which projects a UVA light beam onto a patient's eye at a predetermined distance from the optical head. This system may be used together with a photosensitizer such as riboflavin for strengthening treatment of the cornea.

The projection optics in one embodiment are configured to provide a distance of the patient's eye from the optical head of approximately three inches or 75 mm. The image projection system provides a larger working distance than prior corneal treatment systems and devices. Other working distances may be provided in alternative embodiments. The increased working distance between the optical head and patient's eye provides improved physician visualization and better access to the eye during treatment, for example to add more photosensitizer drops or for other treatment aids.

In one embodiment, at least one UV transmissive liquid light guide connects the light source to the treatment head. This produces homogeneous light distribution. Multiple liquid light guides or a bifurcated light guide may be provided in bilateral systems. Liquid light guides are also more efficient in transmitting light and provide cold light, avoiding the potential problem of hot spots. The flexible light guides also provide for variation in optical head spacing in a bilateral system, and allow for 3 D movement of the optical head or heads if desired.

In one embodiment, the light source in the light source unit is a multi-spectral light source that is filtered to give a narrow band of excitation energy within the UVA/blue light spectrum, and is controllable to provide output light in at least two different wavelengths. The light source may be a short-arc lamp such as a mercury or mercury halide lamp or a short-arc xenon lamp, which emits UVA light as well as light in other wavelengths. Such light sources are more stable than the LED sources used in prior art corneal treatment systems. The light source unit includes an optical system which isolates light to a light beam in the wavelength required for treating the patient and provides the isolated light beam to the light guide for transmission to the optical treatment head. The optical system in one embodiment comprises a focusing device for focusing radiation from the lamp along an optical path and a beam isolating assembly in the optical path which is configured to direct light in a selected wavelength range into the first end of the light guide. The beam isolating assembly may comprise a reflective dichroic mirror which reflects light in the UVA/blue range of around 340 nm to 470 nm and passes other radiation emitted by the lamp, and a filter in the path of reflected light from the mirror which directs light of a predetermined wavelength or wavelength band into the light guide.

The light source unit may selectively provide light at two different wavelengths for treatment purposes, specifically light in a UVA band and light in a blue or blue-violet band. In one embodiment, two different filters are provided which can be selectively positioned in the light path, allowing selection of excitation energy in the UVA band at 365 nm, or a narrow band of blue-violet radiation at 405 nm. The option of UVA or blue radiation allows the surgeon flexibility in achieving different depths of penetration into the cornea for the excitation light. For example, the molar extinction coefficient of riboflavin at 365 nm is about 10,000 and at 405 nm, the extinction coefficient is about 8000. If the riboflavin in the cornea is 0.003 molar, the 365 nm radiation deposits about 75% of its energy to the riboflavin in the first 200 microns of the stroma, whereas with the 405 radiation only about 68% of the beam is absorbed in the first 200 microns. The blue light delivers more energy in the deeper stroma for deeper cross-linking. For thin corneas, the UVA may be preferable since the energy is absorbed more quickly and less energy reaches the endothelium. For thicker corneas, blue light may be used to penetrate deeper into the cornea. From clinical experience treating thousands of patients with prior art photochemical treatment systems using 365 nm radiation, deepithelialization and 0.1% riboflavin soaking, it is known that the cross-linking only occurs to a shallow depth of about 200 microns. Damage (apostosis) occurs deeper, at about 300 microns. The blue light option of this system allows for deeper cross-linking if the surgeon determines deeper cross-linking is more appropriate. Currently marketed systems use monochromatic LEDs and do not allow for selectable excitation wavelengths.

The system may be monocular, with a single optical treatment unit including the optical treatment head, or bilateral, with two optical treatment units adjustably mounted on a support stand for treatment of both eyes simultaneously. The optical treatment unit may comprise only the optical treatment head configured to focus a UVA or blue light beam on a patient's eye, or may incorporate additional treatment or monitoring devices. The optical treatment units may be identical but may be separately mounted to allow for adjusting the separation between the units. Alternatively, more than two treatment units may be provided. The optical treatment unit mount may allow for angular adjustment as well as for adjustment of separation between the optical treatment heads and distance from the eye, to allow for angular variations as well as distance variations of the treatment beams. The light guide from the optical source unit may be bifurcated to provide two separate light guide portions which direct light into the respective optical heads. Excitation light is projected onto the cornea and does not require collimation. In one embodiment, selective irradiation patterns may be provided by the use of pre-prepared reticles printed onto polyester plastic. These prepared reticles have apertures providing a variety of different light distribution patterns and sizes desired by the physicians. An intensity adjustment device may be provided at any suitable point between the light source and optical treatment head for adjusting the intensity or irradiance of the light beam emitted by each treatment head independently. The system may also provide for physician selection of continuous or discontinuous illumination, as well as selection of the on-off time period for discontinuous illumination or fractionation.

According to another aspect, a device and method is provided for applying red light phototherapy to the eye before, at different times during, or after the corneal strengthening treatment. This may reduce the effects of collateral oxidative damage to the eye caused by UVA/blue light and unwanted reactive oxygen species (ROS) from the photosensitizer, primarily hydrogen peroxide which is the major ROS produced in hypoxic environments. This protective effect is accomplished by red light phototherapy that can be employed at different times. If applied before the procedure, the phototherapy builds up anti-apoptotic stores of ATP. If applied during the photochemical therapy, the light can reduce the intracellular superoxide anion production in the mitochondria. If used post-procedure to accelerate the wound healing response, this effect of red light has been demonstrated to be anti-apoptotic for cells exposed to oxidative stress and is used by the military on the battlefield to accelerate wound healing. The red light treatment may be used for reduction of pain in corneal procedures and in other ocular surgery procedures, such as photorefractive keratectomy (PRK), cataract surgery, glaucoma surgery, and the like. In one embodiment, a red light source such as a red light emitting diode, is mounted in the treatment unit adjacent the optical treatment head.

In one embodiment of a monocular or bilateral system, each optical treatment head includes a control system for accurately centering the treatment head and positioning the treatment head at the desired distance from the eye. In one embodiment, distance from the optical projector to the eye is maintained by two laser devices such as laser diodes providing angled aiming beams. The aiming beams may also double as red light phototherapy sources by means of a holographic diffuser on a mechanical slide. In one embodiment, the optical heads and aiming devices are on an articulating arm that can be raised or lowered by a screw mechanism on the arm, and aiming devices are configured so that the two beams coincide when the optical head is at the proper distance from the cornea. In one embodiment, a slide mechanism provided on the optical head allows the diodes to be aimed though attenuation filters for setting the proper distance, and then the slide can be manually activated to allow the beams to pass through holographic diffusers for phototherapeutic uses of the diodes. When the mechanical slide is in the aiming beam position, the aiming beams are small, ⅛ inch circular spots that coincide in the center of the cornea when the distance is properly set. These aiming beams are reduced in intensity to a few microwatts by the attenuating filters. When the mechanical slide is set to the phototherapeutic position, the beams are expanded to about 12 mm in diameter and comprise about 5 mw/cm² of intensity. The operation of the aiming beam laser diodes can be performed manually by a switch on the mobile pole stand or controlled by the microprocessor in the illumination source.

Another feature of the aiming beams at the very low intensity and brightness afforded by the attenuation filters on the mechanical slide is that, if phototherapeutic radiation is not used during the procedure, the aiming beams provide a comfortable alignment or fixation light for the patient to avoid wandering eye movements during periods of discontinuous illumination. When UVA light, for example, is projected onto the cornea the patient can view a violet blue fluorescence caused by the interaction of the invisible UVA with the coating on the projecting lens. During periods of discontinued illumination, the light source is no longer visible and the patient's eye naturally wanders from the desired position unless a fixation or target light is available to keep the patient's eyes correctly aligned. The alignment light sources perform three functions; determining the proper height from the projection optics to the cornea, phototherapeutic radiation for limiting apoptosis, and acting as a fixation or target light to prevent or reduce eye wandering and poor alignment during discontinuous illumination of the excitation energy, i.e. periods during which the excitation light source is not visible. Such eye wandering during the off periods of discontinuous illumination is difficult to monitor and can result in inability to deliver a reliable dose or irradiance, and it is therefore desirable to provide a fixation or target light to reduce the tendency for wandering eye movements and thus improve alignment accuracy during successive treatment periods if discontinuous illumination is selected. In this embodiment, the aiming beams can provide this function in addition to the other functions of proper positioning of the optical heads and the ability to provide phototherapy as a red light source if desired.

According to another aspect, the system includes a device for measurement of the riboflavin concentration in the cornea so the physician can adjust the intensity to achieve the maximum depth of penetration while still reducing the risk of damage to the endothelial cells. In one embodiment, an optical collection device is mounted adjacent to the optical head and is configured to collect photoluminescent emissions from the cornea during treatment. The output of the optical collection device is connected to a photoluminescence monitoring unit. Knowledge of the amount of photoluminescence may help the physician to adjust the treatment to reduce the potential loss of endothelial cells by excess UV radiation. Such cell loss may be a function of a low concentration of the riboflavin, inappropriately high radiation intensity, or both. Excessive riboflavin in the cornea can prevent significant amounts of UV from reaching the endothelial cells, but may also limit the cross-linking depth to the anterior portion of the stroma. Measurement of riboflavin concentration allows the physician to monitor for excessive riboflavin during the procedure and to take appropriate steps to mitigate such conditions. The measurement of riboflavin concentration may be based upon the detection of the photoluminescence of the riboflavin as it interacts with the excitation light. Photoluminescence is an inclusive term given to the combined radiation given off by the fluorescence of riboflavin and the radiation given off as phosphorescence from the triplet state of riboflavin. The emission intensity of the photoluminescent radiation is a function of the light wavelength, the light intensity and the concentration of the riboflavin. Since the wavelength and intensity of the applied light is known, the emission intensity of photoluminescent radiation from the patient's eye (as determined by the photoluminescence monitoring unit and a suitable microprocessor receiving the output of the monitoring unit) is used in one embodiment to measure the riboflavin concentration. In another embodiment the riboflavin may be measured using colorimetry to determine the concentration. A riboflavin concentration measured in either way may be provided to the physician on a display unit associated with the system, and the physician may adjust the treatment light intensity or wavelength, switch to discontinuous irradiation, or take other steps in response to detected reduction or increase in concentration of riboflavin.

According to another aspect, the system for corneal treatment may include a microprocessor-controlled shutter, which is placed in the light beam from the light source, so as to provide discontinuous illumination of the cornea. The on and off times for the discontinuous illumination of the cornea can be determined from the riboflavin concentration readings obtained by photoluminescence measurements as discussed above. This allows for monitoring and control of singlet oxygen creation, which is an important factor in the photochemical cross-linking process in the cornea. The rate at which singlet oxygen is produced varies with local tissue oxygen concentrations as well as the riboflavin concentration and light intensity. For a given riboflavin concentration and excitation energy, the efficiency in producing singlet oxygen vs. hydrogen peroxide is biphasic according to the local molecular oxygen content. Since the oxygen at the surface of the cornea is being consumed at a high rate (75% of the energy is used in the first 200 microns using the current standard methodologies) the posterior stroma does not reoxygenate well and receives little cross-linking, which may result in unnecessary cellular damage. Discontinuous illumination helps to overcome this problem. When the light is shut off, the oxygen consumption by the riboflavin triplets stops and the cornea can reoxygenate from the tear film. High riboflavin concentrations dictate that the on/off times of the fractionation cycle should favor more off time (60% off and 40% on) whereas lower riboflavin concentrations should favor more on time (60% on and 40% off). In order to reduce the risk of the posterior stromal tissue becoming hypoxic, discontinuous illumination may begin after 15 seconds of the initial illumination procedure. It is estimated that, at this point of irradiation, the partial oxygen pressure of the posterior stroma is still around 8-10 torr of oxygen with a high singlet oxygen quantum yield.

Another method of determining the on/off ratios for the discontinuous illumination cycles is to monitor the triplet state phosphorescence in relation to the riboflavin fluorescence. As the ratio of 605 nm/525 nm light drops this indicates that the quantum yield of the triplet state molecules is dropping, and this can signal a drop in the partial pressure of the available oxygen.

The system may also include accessories and methods to improve the penetration of riboflavin through the epithelial barrier to avoid the surgical complications that arise from deepithelialization, which may result in more disruption of the tissue than is necessary for the procedure to be effective. The reduction in patient discomfort combined with more rapid restoration of visual acuity make a transepithelial procedure better for the patient.

The foregoing systems and methods allow the physician to better monitor the patient's eye during surgery. Some embodiments allow monitoring of critical variables during treatment as well as variation of the treatment criteria, for example switching between UVA and blue or blue-violet light, varying the light intensity, varying the beam shape and size, applying red light phototherapy, and using a discontinuous illumination cycle. Another advantage of the system is that distance of the optical head from the eye can be accurately controlled. The system is easy to set up and use, and allows a high degree of control and customization of treatment to a specific patient.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a perspective view of one embodiment of a corneal treatment apparatus and system;

FIG. 2 is a block diagram illustrating the optical source unit of FIG. 1;

FIG. 3 is a cross-sectional view of the optical treatment head of FIG. 1;

FIG. 4 is a perspective view of an embodiment of a bilateral corneal treatment system or photochemical treatment and monitoring system;

FIG. 4A is a functional block diagram of the system of FIG. 4;

FIG. 5 shows a top layout view of the illumination source unit of FIG. 4 illustrating key components;

FIG. 6 is a view of the front control panel of the illumination source unit of FIG. 5;

FIG. 7 is a top plan view of the bilateral optical treatment units and adjustable mounting assembly of FIG. 4;

FIG. 8 is a layout view of the intensity adjustment module of FIGS. 4 and 4a;

FIG. 9 is a frontal cross sectional view of one of the multi-component optical treatment units of FIGS. 4 and 7;

FIG. 10 is a side cross-section view of the treatment unit of FIG. 9;

FIG. 11 is a bottom view of the treatment unit of FIGS. 9 and 10;

FIG. 12 is a front cross sectional view of a modified optical treatment unit which may be used in place of the optical treatment unit of FIGS. 9 to 11;

FIG. 13 is a component layout view of one embodiment of a photoluminescence monitoring module in the system of FIGS. 4 to 11;

FIG. 14 is an example of a kit of optical masks having different aperture shapes and sizes for producing different light patterns or spots that may be projected onto a patient's eye;

FIG. 15A is a cross-sectional view through an eye with a normal cornea;

FIG. 15B is a cross-sectional view through an eye affected by keratoconus offset from the central axis of the eye;

FIG. 16 is a front perspective view of bilateral optical treatment units with an adjustable monitoring assembly similar to that of FIGS. 5 and 7 but modified to provide XYZ or universal positioning adjustment of each optical treatment unit;

FIG. 17 is a front perspective view of the optical head assembly of FIG. 16 but with both optical treatment heads tilted at an angle;

FIG. 18 is an enlarged perspective view of part of optical head assembly of FIGS. 16 and 17 with both optical treatment heads facing downwards;

FIG. 19 shows a top view of one embodiment of a dodging tool for use in evaluating lateral riboflavin dispersion in conjunction with fluorescent intensity monitoring; and

FIG. 20 is a graph of data collected using diluted riboflavin samples and fluorescent intensity raw data measurements.

### DETAILED DESCRIPTION

Certain embodiments as disclosed herein provide for an ophthalmologic or corneal treatment system, apparatus and method.

After reading this description it will become apparent to one skilled in the art how to implement the invention in various alternative embodiments and alternative applications. However, although various embodiments of the present invention will be described herein, it is understood that these embodiments are presented by way of example only, and not limitation.

FIGS. 1 to 3 illustrate one embodiment of a corneal treatment system 100. The system basically comprises an illumination source unit 102 and an optical treatment head 104 which receives a radiation output signal from the source unit 102 via a first flexible elongate light guide or optical cable 105 connected to the intensity adjustment module 106, and a second flexible light guide 107 extending from module 106 to the treatment head. The optical treatment head 104 is mounted on an articulated arm assembly or adjustable mounting device 108, and the arm assembly 108 is adjustably mounted on a support stand 25 via mounting device 108 for adjustment of distance of the optical head from a patient's eye. Arm assembly 108 has a first arm 109 slidably mounted at one end on the stand 25, and a second arm 111 pivotally mounted at the end of arm 109. Each arm can be locked in position when the optical treatment head 104 at the end of arm 111 is at a desired position relative to a patient's eye. The intensity adjustment module or unit 106 is also mounted on stand 25. The illumination source unit 102 is shown separate from the stand but may also be mounted on stand 25 in alternative embodiments, and units 102 and 106 may be combined in a single housing. In the illustrated embodiment, the support stand is a vertical pole with a base including casters or wheels, but other support stands of different configurations may be used in alternative embodiments.

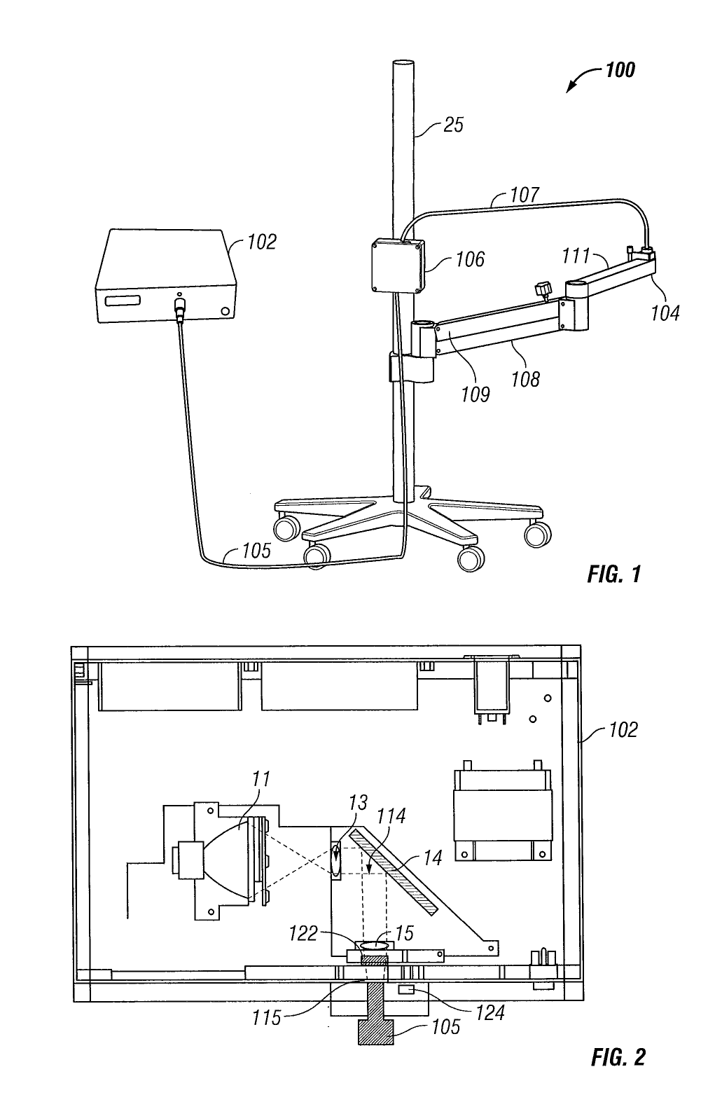

FIG. 2 illustrates the components of the illumination source unit 102 in more detail. Unit 102 comprises an outer housing containing a light source 11 and an optical system 114 which directs radiation emitted by source 11 along a light path from the source to an output 115 to which the inlet end of light guide 105 is connected. The light source may be an ellipsoidal reflector short-arc lamp which emits multi-spectral light including UVA light in a wavelength desirable for corneal photochemical treatment purposes. Some examples of suitable light sources are 100 watt short-arc mercury or mercury halide lamps or xenon lamps, or LED light engines. Optical system 114 comprises a collimating lens 13 which directs light emitted by lamp 11 onto a wavelength control device or limiter comprising a dichroic 45 degree turning mirror 14, and a focusing lens 15 which receives light reflected from mirror 14 and directs the light through filter 122 into the light guide, so that the wavelength of light is directed to output port 115. The dichroic mirror is a UVA reflective mirror which reflects at least UVA light while transmitting infrared radiation so it is not directed into the light guide. The infrared radiation which passes through mirror 14 may be collected by a suitable absorber or heat sink (not illustrated). Filter 122 in one embodiment is a UVA filter that acts to isolate the light directed into light guide to a desired light treatment frequency, and has a 10 nm bandwidth (FWHM) at 365 nm. A manually operable shutter is actuated by control knob 124 on the front of the light source unit to block the path of light through outlet port 115, allowing for emergency manual cut-off of light delivery to the light guide 105. Filter 122 may be located in another part of the system in other embodiments, for example, in intensity adjustment module 106 or in treatment head 104.

The mobile pole stand 25 with the adjustably mounted articulating arm 108 allows for easy positioning of the optical head over the patient's eye. In this embodiment, the system is monocular and has a single optical treatment head 102 used to treat one eye at a time. However, a second optical treatment head may be provided in other embodiments for simultaneous or bilateral treatment of both eyes. Adjustment knobs are provided for vertical and horizontal adjustment of the location of the light emitting end of the optical treatment head.

Intensity adjustment module 106 controls the intensity of UVA light transmitted along light guide 107 to the treatment head, and has a manual controller to allow a physician to control treatment light intensity. In one embodiment, the controller adjusts the separation between an outlet end of light guide 105 and an inlet end of light guide 107, thereby varying the output intensity at treatment head 104.

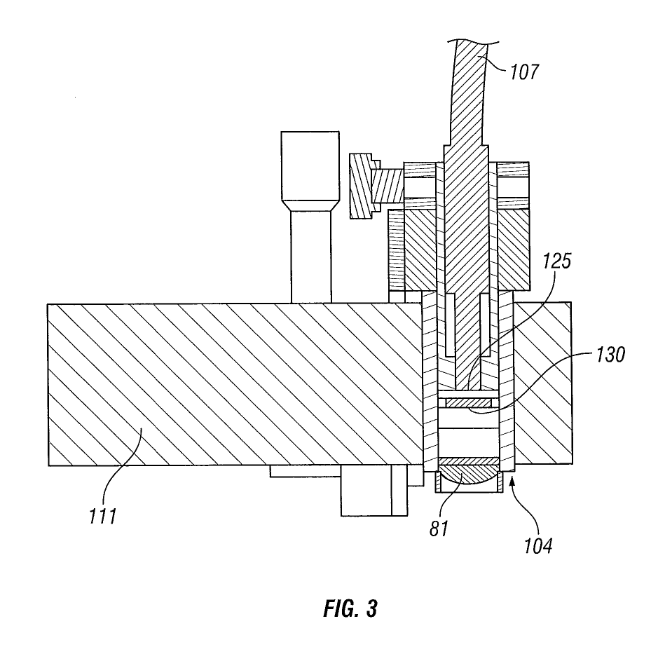

Treatment head 104 is mounted at the end of arm 111, as illustrated in more detail in FIG. 3, and incorporates an optical mask or reticle 130 for controlling the size and shape of the beam and a projection optic or lens 81 which is located at the exit end of the treatment head. The exit end 125 of light guide 107 is secured in the treatment head and directs UVA light onto optical mask 130. In one embodiment, mask 130 has a central circular opening and the mask and lens are configured to focus a circular spot of predetermined diameter at a predetermined working distance from the exit end of the optical treatment head. Different masks may provide for different beam diameters in a suitable range to cover a normal range of eye dimensions, as described in more detail below.

In one embodiment, the system illustrated in FIGS. 1 to 3 is used in a corneal treatment method using riboflavin as a photosensitizer and UVA light at an excitation wavelength of around 365 nm as the excitation energy for photochemical corneal cross-linking.

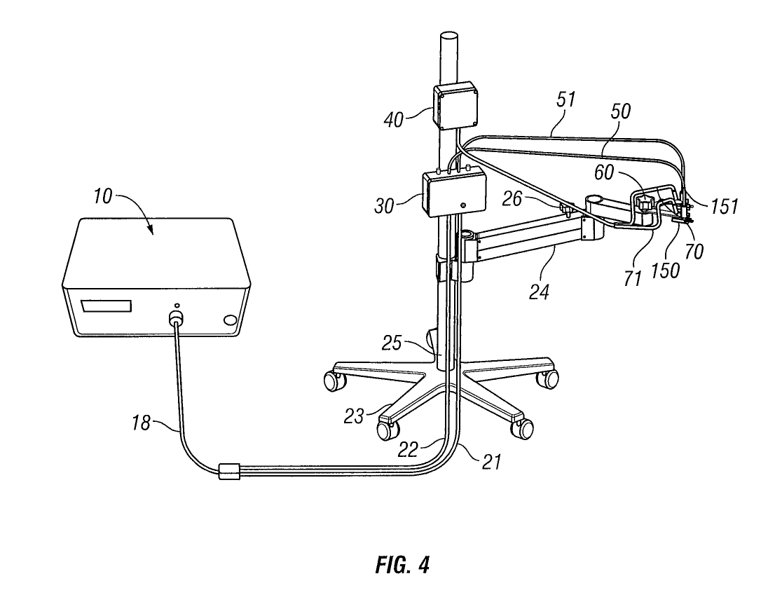

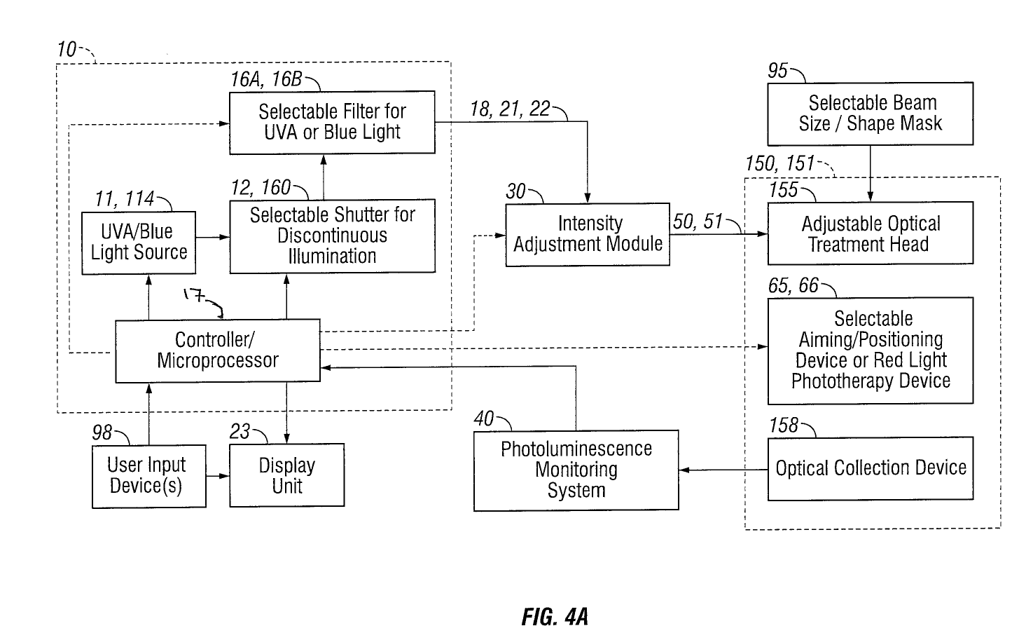

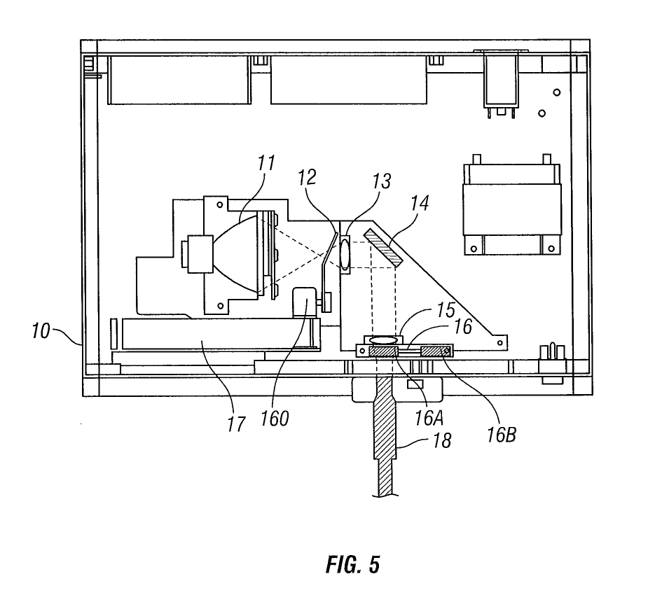

FIGS. 4 to 13 illustrate a second embodiment of a bilateral system for photochemical ocular treatment such as corneal collagen cross-linking using riboflavin as a photosensitizer. Some components in this system are identical to components of the monocular system of FIGS. 1 to 3, and like reference numbers are used for like parts as appropriate. In this embodiment, UVA/blue light is used for the excitation energy. Referring to FIGS. 4 and 5, an illumination source unit 10 contains a multi-spectral light source 11 that delivers a user-selected excitation wavelength to bifurcated, UV transmissive liquid light guide 18. The light guide splits into separate light guide outputs 21 and 22 that are connected to illumination intensity adjustment module 30 mounted on a mobile pole stand comprised of pole 25 mounted on a base 23 with casters. Other support stands of different configuration may be used in place of pole 25 with base 23 in alternative embodiments. Outputs of module 30 are connected by light guides 50, 51 to respective left and right optical treatment devices or units 150, 151. The right treatment device 151 is described in more detail below in connection with FIGS. 9 to 11. The left treatment device 150 is identical to the right treatment device 151.

The pole allows attachment and vertical positioning of an adjustable mounting mechanism including articulating arm 24 on which the treatment devices 150, 151 are mounted, and provides mounting points for illumination intensity adjustment module 30 and an optical monitoring module 40. Modules 10, 30 and 40 may be combined in a single unit in other embodiments. The illumination source unit 10 is shown as separate from the mobile stand but can be affixed to the stand in another embodiment. The distal end of articulating arm 24 carries the two optical treatment devices or units 150, 151 on laterally adjustable arms 62, 64, each treatment device including an optical treatment head 155 for projection of patterns of excitation light onto the patient's eyes, in addition to other components described in more detail below in connection with FIGS. 9 to 11. In one embodiment, light guides 18, 21, 22, 50 and 51 which conduct the excitation energy to each optical treatment head are liquid light guides, because the water-based liquid in the light guide absorbs infrared radiation from the lamp source that could adversely affect tissues. Liquid light guides generally have greater transmission efficiency for UV and visible light than fiber bundles while providing greater flexibility to allow for adjustment of the position of each treatment unit. An additional benefit of using liquid light guides is that they are effective in homogenizing light beams collected from non-homogeneous light sources or reflectors.

FIG. 5 illustrates the layout of the illumination source assembly with an ellipsoidal reflector short-arc lamp 11 as the light source, as in the first embodiment. In one embodiment this lamp is a 100 watt short-arc mercury or mercury halide lamp. In a different embodiment, this lamp is a 100 watt short-arc xenon lamp that is characterized by a lower UVA output and a greater continuum of high intensity blue wavelength light. Microprocessor 17 controls the opening and closing of shutter 12 that either blocks or allows passage of radiation emitted from the lamp. Shutter 12 is a mirrored aluminum material to reflect radiation away from the optical path. The reflective quality of the material prevents a heat build up on the shutter and potential transfer of heat to the connecting solenoid assembly. The shutter is affixed to a rotary solenoid 160 to affect the opening and closing operation. Rotary solenoids are high reliability components with normal lifetimes exceeding 1 million cycles. When shutter 12 is opened, the light from the lamp reflector is collected by collimating lens 13 and directed to dichroic 45 degree turning mirror 14 that reflects UVA and blue light in a wavelength range of around 340 nm to 470 nm, while passing infrared radiation. The reflected light from the mirror is collected by focusing lens 15 and directed through one of the filters on filter assembly 16 into the input of bifurcated light guide 18. Filter assembly 16 is on a slide mechanism connected to an actuating switch on the front panel. Two narrowband bandpass filters 16A, 16B are mounted on the optics filter assembly 16 and an actuating switch position determines which bandpass filter is placed in front of the light guide. In one embodiment, filter 16A is a UVA filter that has a 10 nm bandwidth (FWHM) at 365 nm and filter 16B has a 10 nm bandwidth (FWHM) at 405 nm. Such filters are commercially available from various optical suppliers.

Allowing for selection of the wavelength of the excitation light is an important option for the user because the wavelength determines the depth of penetration of the light into the riboflavin soaked cornea. This in turn affects how much cross-linking may be done at different depths of the corneal stroma. The molar extinction coefficient of riboflavin is 10,066 cm⁻¹/M at 365 nm but the molar extinction coefficient of riboflavin is only 7884 cm⁻¹/M at 405 nm. It is known from the Beer Lambert law that, for a given wavelength and excitation energy, the fluorescent intensity of a sample is linearly proportional to the concentration of the fluorophore, in this case riboflavin. Calculation of the light absorption by riboflavin at various depths of the cornea of the two wavelengths is possible using the Beer Lambert equation. In this equation A=2−log₁₀% T, where A is the absorbance of energy by a chemical fluorophore and T is the transmission. The Beer Lambert law states that A=Ebc where E is the molar extinction coefficient for a particular chemical and b is the path length of the measurement and c is the concentration of the chemical. For a 0.1% solution of riboflavin at a depth of 500 microns the absorption value at 365 nm is calculated as A=1.10. The value of A for the same solution and path length for 405 nm radiation is calculated as A=0.86. From the formula A=2−log₁₀% T it is shown that 64% of the incident energy of 365 nm radiation is absorbed by riboflavin in the first 200 microns of the cornea. The same calculations at 405 nm indicate only 55% of the radiation is absorbed by the riboflavin in the first 200 microns of the stroma. If the user determines that it is desirable to cross link deeper into a cornea, they have the option to select a more penetrating radiation like 405 nm by positioning filter 16B in the light path from unit 10. They may select a less penetrating wavelength like 365 nm for shallower cross-linking, by positioning filter 16A in the light path. No commercial cross-linking devices are known that offer such wavelength selection to the user. It is known from the literature that the use of 0.1% riboflavin and 365 nm radiation only cross links collagen about 200-250 microns into the anterior stroma, and the option for a wavelength to cross link deeper is therefore a desirable feature.

An additional potential advantage of the 405 nm wavelength is the option to use less intense light to accomplish the same amount of cross-linking. The production of singlet oxygen by excited riboflavin triplet molecules is related to the number of incident photons, not the energy of the photons. Riboflavin is excited at both 365 nm and 405 nm to its higher energy states. By the formulation E=hv it is determined that a 405 nm photon is 10% less energetic than a 365 nm photon, and that to have equivalent stoichiometric reactions at 405 nm and 365 nm the incident blue light fluence is reduced to 90% of the UVA value.

Another additional feature of the blue light option for excitation energy is that the lower absorption of blue light by riboflavin in the anterior cornea translates into less oxygen consumption in the anterior stroma, and this may allow better reoxygenation of the posterior stroma, as discussed in more detail below.

FIG. 4A is a functional block diagram of the various components of the multi-functional treatment system of FIG. 4. As illustrated in FIG. 4A and FIG. 7, each multi-functional treatment device 150, 151 mounted on the pivoted end portions of the articulating arm includes the adjustable optical treatment head 155 as well as aiming devices 65, 66 and an optical collection device 158 which are described in more detail below. The treatment head 155, aiming devices 65, 66 and optical collection device 158 are all mounted on a common support or mounting plate 154 (see FIGS. 9 to 11) which may comprise an integral end portion of the respective arm 63 or 64, or may be a separate support or base mounted on the arm. Although FIG. 4A illustrates only one treatment device, it will be understood that a second identical treatment device is provided and the output of intensity adjustment module 30 is connected to both optical treatment heads 155, as illustrated in FIG. 4. Additionally, the output of both optical collection devices 158 is connected to photoluminescent monitoring system 40, as described in more detail below.

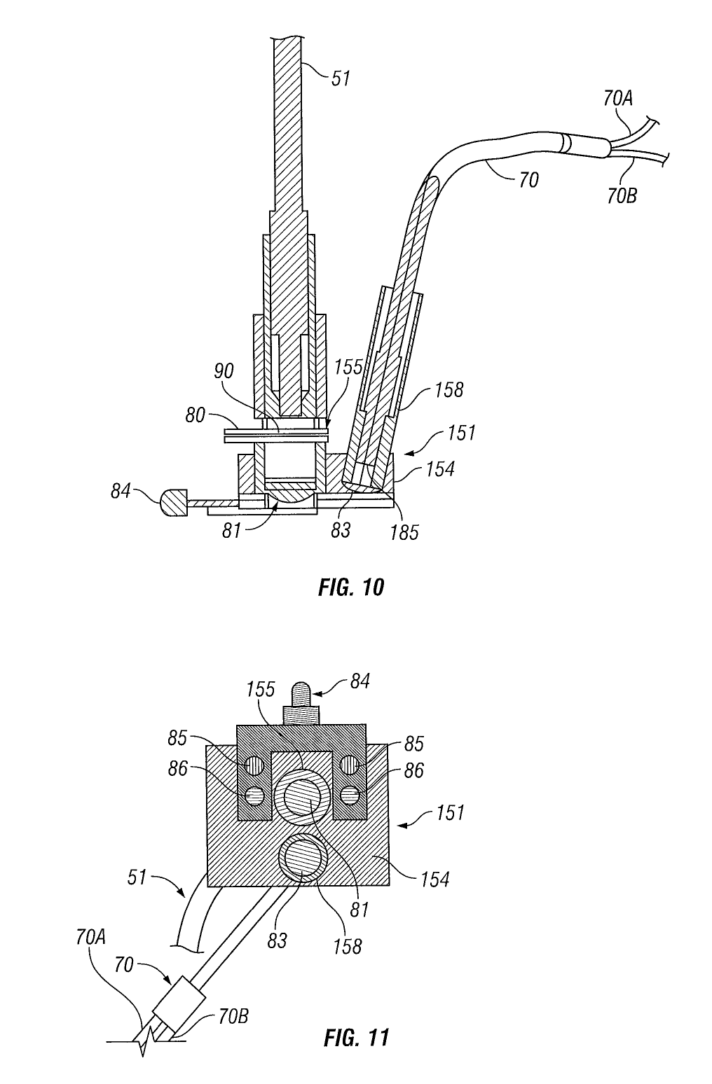

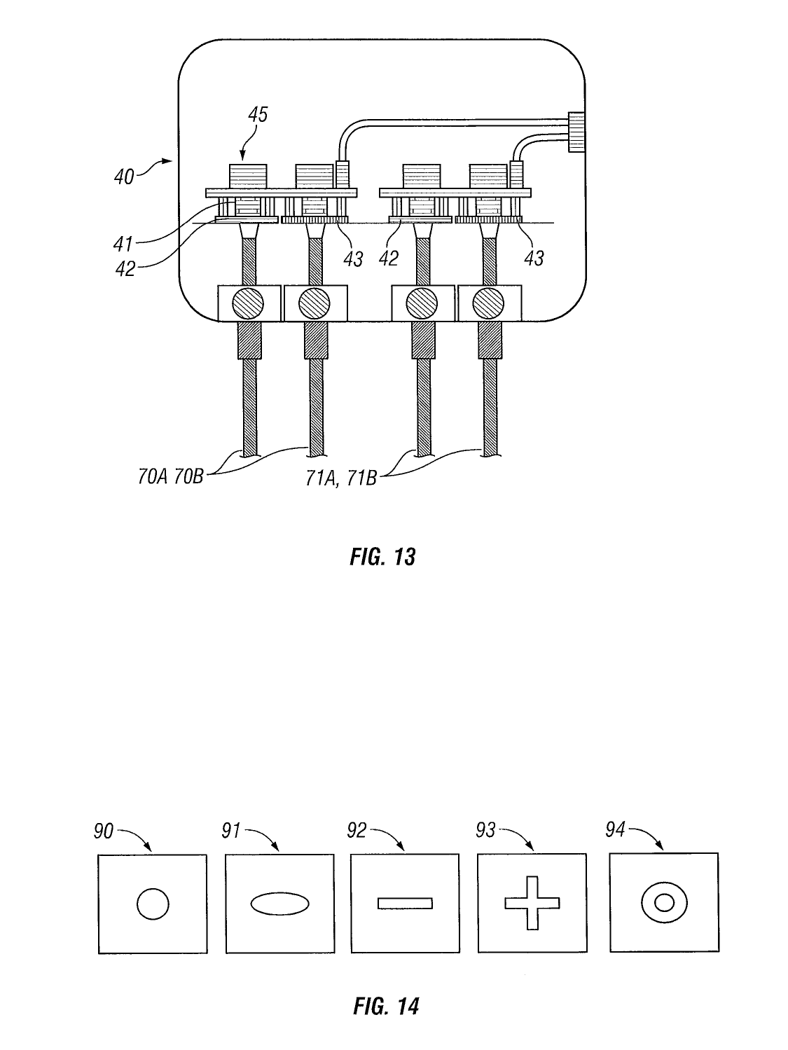

Each optical treatment head 155 is adjustable to produce a light beam of selected frequency in different treatment sizes and shapes using optical masks selected from a kit 95 of masks with different aperture patterns, as described in more detail below in connection with FIG. 14. The selectable UVA or blue light source unit 10 includes a selectable shutter 12 which can be used for discontinuous illumination via solenoid 160. Discontinuous illumination may be controlled by microprocessor or controller 17 automatically or via user input. A user input device 98 is provided for operator control of various operating parameters, for example whether to use continuous or discontinuous illumination and the time period for discontinuous illumination. A display unit 23 connected to the microprocessor 17 displays operational parameters of the system to the operator. As indicated in FIG. 4A, the aiming devices 65, 66 may be dual function devices which can also be used as red light phototherapy devices, as described in more detail below in connection with FIGS. 9 and 11. Alternatively, treatment devices or units 150, 151 may include separate treatment heads for use in red light phototherapy. The output of each optical collection device 158 is connected to the photoluminescence monitoring system 40, which provides output signals proportional to the intensity of detected phosphorescence and fluorescence emissions from the irradiated cornea. These output signals are connected to the controller 17, which displays photoluminescence information on the display unit, as discussed in more detail below in connection with FIGS. 10 and 13.

Various adjustable features of the system described below involve manual input by an operator at the various units in order to vary operating conditions, such as intensity adjustment via module 30, selection of the aiming or phototherapy function of devices 65, 66, selection between the UVA or blue light filters 16A and 16B, and positioning of the optical treatment heads. In an alternative embodiment, these features may be adjusted by an operator by input at remote input device or keyboard 98, and the controller in this alternative has control outputs to the selectable filter assembly 16A, 16B, intensity adjustment module 30, and the selectable aiming or phototherapy devices 65, 66, as indicated by the dotted lines in FIG. 4A. This provides for more easy control of the system by the operator. An automatic emergency shut off feature may also be provided.

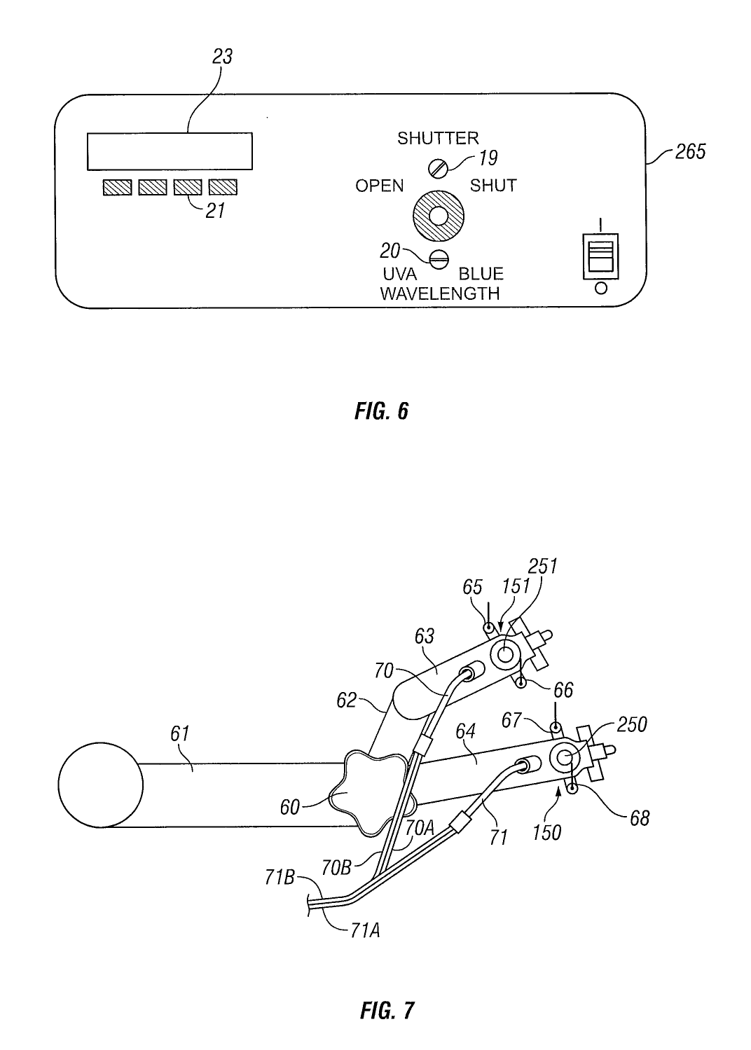

FIG. 6 illustrates one embodiment of a control panel 265 provided on the front of illumination source unit 10 including user input devices and display unit 23. In other embodiments, the controller may be a stand alone desk top or lap top computer, or a personal digital assistant or the like, with a standard display unit and a keyboard input device for user input control selections for the various selectable control parameters of the system, which may be transmitted by wired or wireless communication signals to control various system components. Panel 265 has a manual wavelength selection control switch 20 to allow an operator to switch between UVA and blue light, and a manual shutter control switch 19 to switch between continuous and discontinuous illumination. Softkey inputs 21 below display 23 on the panel can be used by an operator to control the shutter cycle. The softkeys are switches that change function as the display changes.

Referring to FIGS. 4 and 7, the mobile pole stand with the mounted articulating arm provides for easy positioning of the optical treatment heads over the patient's eyes. FIG. 7 is an enlarged top plan view of the articulated arm assembly and treatment devices 150, 151 of FIG. 4. The light guides 50, 51 for delivering the excitation energy to the optical heads 155 are not shown in this view for purposes of clarity but the receptacles for these light guides are shown as 250 and 251. The light guides 50 and 51 are shown in FIG. 4 connecting to these receptacles.

The height adjustment knob 26 shown in FIG. 4 provides a vertical adjustment of the arm. Lateral adjustment of the optical heads to accommodate different interpupillary distance is provided by the pivot arms 62, 63 and 64 on the distal end of the articulating arm, as illustrated in FIG. 7. When knob 60 is loosened, both arms 64 and 62 are free to pivot around the center of knob 60 to adjust the interpupillary distance and to align each optical head with the respective eye of a patient. When the optical head on arm 64 is positioned over one eye, knob 60 can be tightened to fix the position of arm 64 in relation to the articulating arm. Arms 62 and 63 are still movable at this point and a combined movement of the two arms can allow for XY axis adjustment of the second optical head over the patient's second eye for bilateral operation. A second adjustment knob (not visible) secures the second optical head in the selected position. An interpupillary distance of 45-75 mm is accommodated with a design length of arm 64 at 4.7 inches. In alternative embodiments, the manual positioning knobs may be eliminated and a remotely controlled drive system may be provided for vertical and horizontal positioning of the treatment heads.

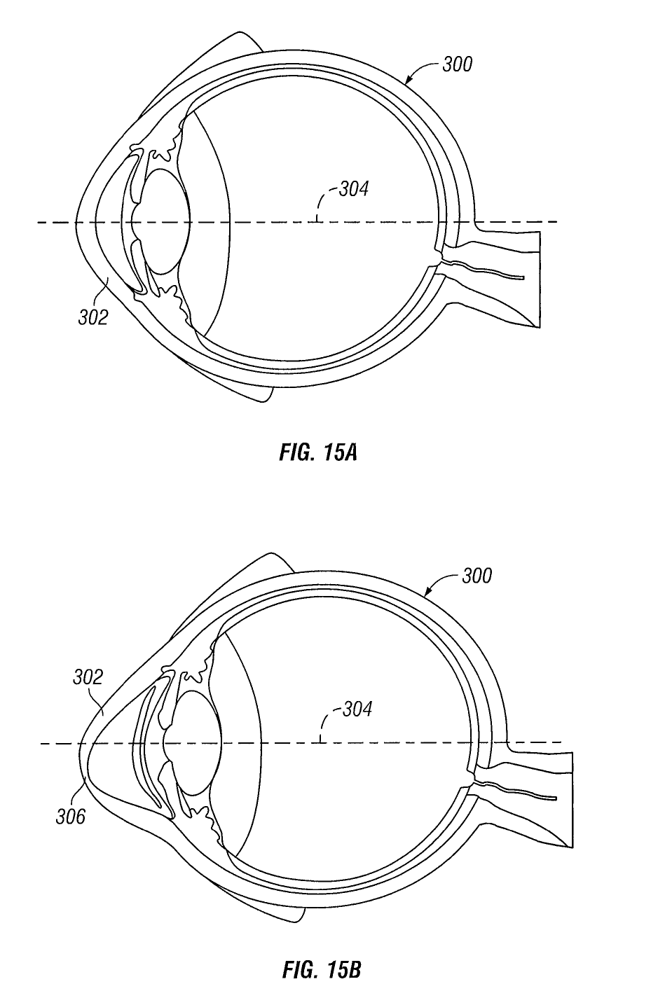

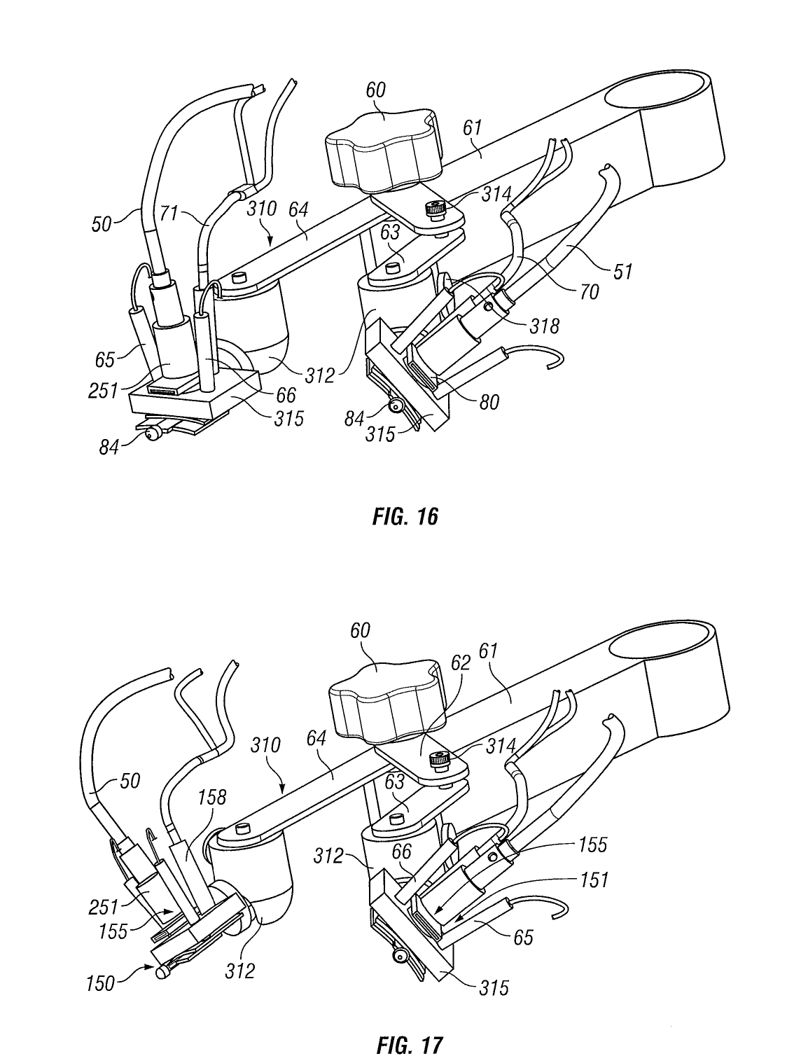

In the treatment system of FIGS. 4 to 14, the treatment light beam of each optical head may be directed concentric to the optical axis passing through the center of the cornea to the center of the lens. It is desirable in some circumstances to position the light beam on an optical axis different than the corneal-lens optical axis. For example, if the apical distortion from keratoconus is in the inferior portion of the cornea, it may be desirable to place the optical axis of the illumination beam concentric with the central axis of the apical distortion to maximize the radiation concentrically around the apical distortion. FIGS. 15A and 15B illustrate one example of the apical distortion of keratoconus compared to a normal cornea. FIG. 15A illustrates an eye 300 with a normal cornea 302, with the dotted line 304 representing the optical axis passing through the center of the cornea. FIG. 15B illustrates eye 300 with keratoconus causing an off-axis conical distortion 305 and resultant thinning of the cornea at 306. This requires an XYZ positioning flexibility for the optical head, and this can be achieved by the mechanical arrangement shown in FIGS. 16 to 18, as described below.

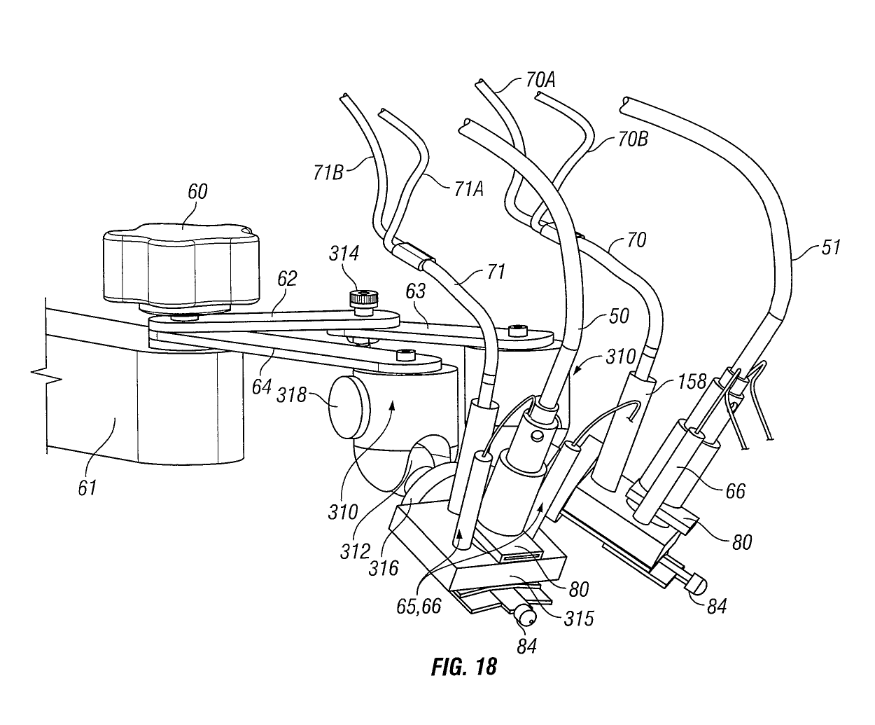

FIGS. 16 to 18 illustrate an alternative mounting assembly 310 for the treatment devices 150, 151 in the system of FIGS. 4 to 14. Mounting assembly 310 provides for individual angular (Z direction) adjustment of each optical head in addition to X and Y directional adjustments. In the embodiment of FIGS. 16 to 18, each treatment device or unit 150, 151 is mounted to the respective support arm via a swivel or ball joint 312, so as to permit angular adjustment of the beam direction, as described in more detail below. All other parts of the mounting assembly and the treatment devices are identical to those of the previous embodiment, and like reference numbers are used for like parts as appropriate. As best illustrated in FIG. 18, mounting plates 315 carrying the optical treatment heads 155 and other optical components are secured via connectors 316 to the respective ball joints 312 secured on the ends of arms 63 and 64. X and Y directional adjustment (height and horizontal position) of the treatment units or devices 150, 151 is provided by vertical adjustment of arm 61 on support stand 25, and articulation of the arms 64 and 62 about the pivot connection to arm 61, as well as articulation of arm 63 about the pivot connection to arm 62. Knobs 60 and 314 are tightened to fix the positions of arms 63 and 64 and the separation between the treatment units, as in the previous embodiment. Freedom of movement in the Z-axis is achieved by the ball joint 312, which can be loosened by releasing the locking knob 318. When loose, the optical treatment unit can be tilted or swiveled forward and backward and/or side to side. When the desired beam angle is achieved, the position can be locked into place by retightening knob 318. The ball joints allow the angle of each illumination beam emitted by the optical treatment heads to be changed anywhere within a conical angle. In one embodiment, the conical adjustment angle is +/−35 degrees from perpendicular, but optical adjustment angles of different ranges may be provided in alternative embodiments. The two heads 155 are adjusted independently dependent on the desired treatment angle for each eye, where both eyes of a patient are to be treated simultaneously. FIG. 16 illustrates one possible orientation where treatment device or unit 150 faces down while treatment device 151 is angled sideways. FIG. 17 illustrates another example where both treatment units are oriented at an angle to direct the treatment beams of the two optical heads 155 at non-vertical angles. In FIG. 18, both treatment units face downwards.

In alternative embodiments, the manual positioning knobs may be eliminated and a remotely controlled drive system may be provided for vertical, horizontal, and angular positioning of the treatment heads. X, Y and Z direction positioning may then be controlled remotely by the operator via a computer input device, touch screen or the like, or may be carried out automatically on entry of patient eye parameters by the physician, for example.

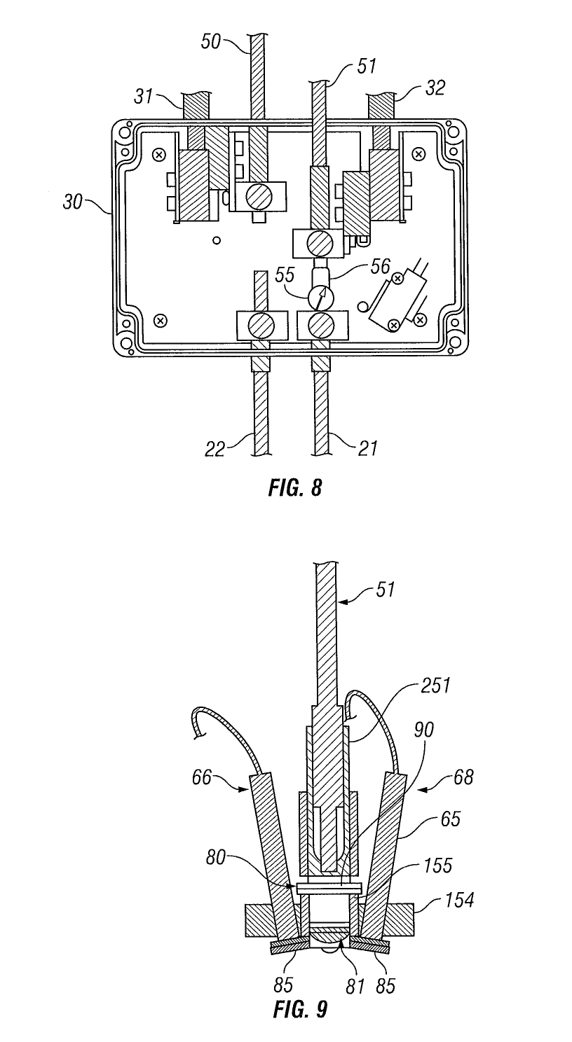

The output light intensity adjustment for each eye in the system of FIGS. 4 to 14 is accomplished using the intensity adjustment module 30 illustrated in layout view in FIG. 8. Mechanical brackets are affixed to the output light guides and these brackets are connected to commercial screw-driven linear slides 31 and 32. The bifurcated input light guide ends 21 and 22 are fixed at the bottom of the module. Turning the externally accessible knobs on slides 31 and 32 clockwise advances the delivery light guides 50 and 51 toward the input light guides and increases the intensity of the output. Likewise, turning the knobs in a counterclockwise direction reduces the intensity. The output is measured by using an external hand held radiometer under the output optics. Appropriate radiometers for UVA or blue light are commercially available from a variety of sources. Adjustment of the output of each optical head within 0.01 mw/cm² is obtained by this design. As an alternative design, adjustable neutral density filters could be placed between the input and output light guides but these filters are often subject to long term UVA deterioration. FIG. 8 illustrates the maximum intensity adjustment for excitation light guide 51 and the minimum intensity adjustment for excitation light guide 50.

In the illustrated embodiment, a manually operable switch 55 allows a user to convert from bilateral to monocular operation. Switch 55 is connected to shutter 56. In the position of shutter 56 as shown in FIG. 8 the light entering from light guide 21 is blocked from entering the delivery light guide 51 and the instrument is set for monocular operation. When the switch is rotated from this position, the shutter rotates out of the light path and closes a microswitch. Light now travels to both output heads and the closed microswitch completes a circuit to light an LED on top of the module alerting the user that the instrument is in bilateral mode. In alternative embodiments, the manual switch may be replaced by a remote control device such as a computer module with a user control input or touch screen for switching between bilateral or monocular operation. The same control input may be used to enter commands to vary other adjustable features of the system, as indicated in FIG. 4A, such as the excitation energy frequency, intensity, continuous or discontinuous illumination, treatment period, treatment head height, separation, and angle, and the like.

One of the optical treatment devices 151 is illustrated in more detail in FIGS. 7 and 9 to 11. As illustrated, each optical treatment device comprises optical treatment head 155 vertically mounted on support 154 at the end of the respective arm 63 or 64, a positioning apparatus comprising a pair of aiming devices 65, 66 each mounted at an angle on opposite sides of head 155, as illustrated in FIGS. 7 and 9, and optical collection device 158 also mounted on support 154 adjacent the optical treatment head 155, as illustrated in FIG. 10. Light guides 50 and 51 deliver the radiation to the optical heads 155 as illustrated in the cross sectional view of one optical treatment head in FIG. 9. In this view the light guide 51 serves as the light projection source for the optical projection system that incorporates reticle holder 80 and projection optic or lens 81. In one embodiment, the projection lens 81 is a 12.7 mm diameter lens with a 25 mm focal length and projects light according to the pattern on the slide at a distance of 75 mm with a magnification factor of 3.

As discussed above, the system includes a kit 95 of slides or masks that offers various circular spot sizes, elliptical shapes on various axes, annular shapes, half moon shapes and horizontal and vertical slit shapes, some of which are illustrated by way of example in FIG. 14. A projection slide selected from the kit 95 of alternative projection slides is inserted in reticle holder 80 and controls the treatment beam size and shape according to the physicians desired irradiation pattern. For example, projection slide 90 is used to project a 9 mm diameter circle of UVA or blue radiation at a working distance of around three inches or around 75 mm from the treatment head for the treatment of the whole cornea in the case of keratoconus. The kit includes slides or masks with different size circular openings for different patient eye sizes, as well as other shape openings for different treatment conditions, as described in more detail below.

Aiming or positioning apparatus 65, 66 mounted in each optical treatment unit 150 and 151 assists an operator in positioning the projection optic or lens 81 at a desired working distance from the cornea. In the embodiment of FIGS. 7 to 11, the aiming devices 65, 66 are laser diodes. The distance of optic 81 from the cornea is determined to be equal to the desired working distance when the two aiming beams from laser diodes 65 and 66 coincide with each other as a single spot on the patient's eye. If the aiming beams do not cross at the eye, the height adjustment knob 26 on the articulating arm can move the optical heads up or down until the beams coincide at the correct position. This provides a more accurate method for positioning the optical heads at a predetermined distance relative to the patient's eyes.

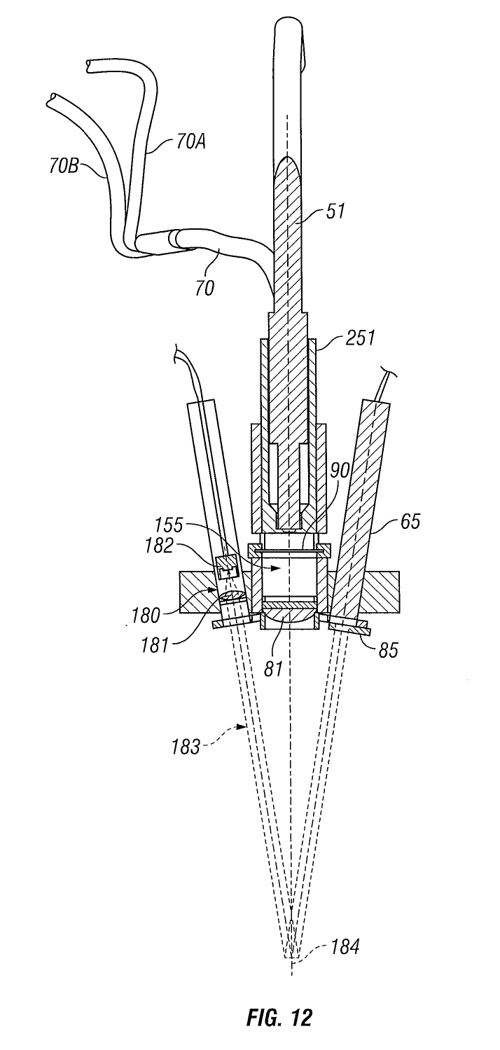

An alternative aiming or positioning apparatus for fixing the distance from the optical head to the cornea is shown in cross sectional view in FIG. 12. In this construction, only one laser diode 65 is used which centers on the cornea at the desired working distance from the optical head, which may be three inches in one example. The second laser diode 66 used in the previous embodiment to give coinciding beams at the desired distance is replaced by an optical assembly 180, which comprises a short focal length lens 181, and a photodiode 182, which replaces the second laser diode normally used to give coinciding beams at three inches. In this configuration, when the remaining laser diode 65 is pulsed, the return signal 183 from the first surface or corneal surface 184 is at a maximum when the distance is three inches from the cornea to the optical head. Photodiode measurement is only made when the laser is pulsed, and the response of the system is independent of all background radiation. The optical assembly focuses the reflected beam from the laser pulse to the active sensing portion of the photodiode, and any off-axis radiation does not hit the sensing portion of the photodiode. The operator can move the assembly up and down until the photodiode reading is a maximum and this occurs at the selected working distance from the center of the aiming beam of the laser diode, which is three inches in this embodiment, although the apparatus (positioning apparatus and focal length of projection optic) may be modified to provide for different working distances in other embodiments, depending on the amount of space needed by a physician. An advantage of this system is that sub-microwatt radiation of any wavelength suitable for detection can be used so that the patient receives less radiant energy into the eye.

The ability to project different light beam patterns allows the user unique flexibility in cross-linking the cornea to accommodate the individual biomechanical architecture. This permits a greater degree of customization of treatment to a particular patient, by selecting a particular beam size and shape as well as selecting the other adjustable treatment parameters noted above on a patient by patient basis. In treating younger patients with keratoconus, a circular pattern with a small 7 or 8 mm diameter may be required. An adult with keratoconus may require a 9 to 11 mm diameter spot. The spot size is selected to reduce or avoid unnecessary radiation being applied to peripheral regions of the cornea, and to apply radiation more accurately to the regions where it is needed. For patients with myopia, an annular projection pattern may be applied, using mask or slide 94 in FIG. 14, in order to flatten the central region of the cornea. In some cases, this may avoid the need for more invasive surgical intervention. The radiation from the light guide passes through a selected plastic slide that may have the radiation pattern printed in black ink on one side, for example. Polyester or OP-4 acrylic may be used as the slide material since the transparency of these materials in the UVA/blue spectrum is quite high. The slides are inexpensive to produce and can be printed using polyester transparency paper on a 600×600 dpi laser printer. The slides are pre-mounted on holders that can be inserted into reticle receptacle 80 on the optical head 155 for each eye. An inexpensive printer and template program can be supplied to the user so they can design and print slides on-site to meet their custom needs.

An elliptical beam shape as illustrated for slide 91 of FIG. 14 may be selected if keratoconus or pellucid marginal degeneration or other corneal condition with a more severe thinning of the stroma in the inferior cornea is to be treated. In such cases, strengthening the cornea on a horizontal plane may help correct the biomechanical strength and corneal curvature in a fashion to provide more optimal visual acuity. In this case the surgeon may cross link using an excitation energy contained in an elliptical beam shape placing the long axis of the ellipse on the horizontal axis of the cornea, for example by using slide 91 of FIG. 14. The specific architectural structure and needs of the cornea vary from patient to patient, and selective biomechanical strengthening is a desirable feature. Currently marketed devices only allow for circular spots of illumination. The system described in the above embodiments provides improved capability for customized treatment based on individual corneal architecture. The distance from the optical delivery system to the eye can be adjusted based on the focal length of the projection lens, so that the beam forms a spot of the desired size on the patient's cornea.

In one embodiment, each optical treatment unit 150, 151 also includes a red light phototherapy device for applying red light phototherapy to a patient's eye before, during, or after photochemical treatment for collagen cross-linking. The red light phototherapy device may be a separate component, or may be integrated with the positioning apparatus, as in the illustrated embodiments of FIGS. 4 to 13 and 16 to 18. In these embodiments, the beams from the alignment laser diodes 65, 66 are directed through selectable filters 85, 86 mounted on a mechanical slide on the bottom of support 154, as illustrated in FIG. 11, so as to switch between aiming beams for optical head positioning purposes and red light beams for phototherapy purposes. When the manual slide is pulled forward, filters 86 are placed in front of the output of laser diodes 65 and 66. Filters 86 are attenuating filters which reduce a nominal 3-mw diode output to a few hundred microwatts of output. The beam shape remains unchanged, and may be a 3 mm dot, but the power is limited. According to IEC60825, the maximum permissible exposure of a coherent beam from 400 nm to 700 nm on the eye is 0.002 J/cm². Although the height alignment by the operator may be completed in 3 or 4 seconds, it is reasonable to design for a 80 second aiming beam alignment procedure. This provides a safety factor for the patient. A 3-mw laser diode operated in pulse mode at a 3% duty cycle has a power output of 0.00009 W, and a 3 min dot has an area of 0.071 cm². In 80 seconds, a total of 0.142 J/cm² of non-attenuated light is delivered from both beams, so an attenuation filter with an optical density of 2 (99% reduction in energy) for filter 86 allows 80 seconds of laser beam alignment at a cumulative dose of 0.0014 J/cm².

When the mechanical slide 84 is moved to the back position, filters 85 are placed in front of the laser diodes. Filter 85 is a holographic circular light shaping diffuser with a 5 degree spread which shapes the laser diode beam to a circular area 10 mm in diameter at 3 inches. This provides laser diodes with a secondary use for providing red light phototherapy to ameliorate oxidative damage done to cells. In this case the holographic light shaping diffusers are 95% efficient in transmission and the laser diodes can be operated at full power giving about 5 mw/cm² for the combined beams over the 10 mm circle, thus providing a therapeutic dose of 3.0 J/cm² in a 10 minute period. In the alternative embodiment of FIG. 12, the holographic diffuser and attenuator filters are only needed for the single emitting laser diode, and not for the optical assembly 180 which receives the reflected signal. In this alternative, either holographic diffuser filter 85 or attenuator filter 86 may be positioned in front of output end of laser diode 65, by suitable positioning of slide plate 84, to allow for a phototherapy function as well as fixing the distance from the cornea.

Red light in the wavelength range of 580 nm-830 nm is known to photodissociate nitric oxide from cytochrome c oxidase. Nitric oxide is a competitive inhibitor of oxygen at this enzymatic site, and when nitric oxide is present no oxidative phosphorylation can occur. Removal of the nitric oxide allows oxygen to bind to the enzyme and ATP production begins. Nitric oxide is produced in quantity during conditions of oxidative stress such as those found during photochemical therapy. Removing the nitric oxide allows the cells to produce ATP energy for repairing the effects of the oxidative stress. Cells with insufficient ATP production are unable to repair the oxidative damage and die either by apoptosis or necrosis. Cellular death initiates wound healing responses that can have many adverse affects in the cornea, such as inflammation of the nerve cells in the anterior cornea, which causes pain and dry eye conditions. Cytokines such as TGF-beta and IL-1 released in response to the cellular deaths may induce differentiation of keratocytes into myofibroblasts that produce Type IV collagen instead of Type I collagen resulting in corneal haze and reduced vision.

The maximum benefits of red light phototherapy are achieved when the treated cells are under oxidative stress. During or after a photochemical treatment the oxidative stress is high so these are occasions when the greatest benefit may be obtained. In some cases like keratoconus there is an ongoing mild oxidative effect associated with the condition and treatment before the photochemical therapy allows these cells to build up advance stores of ATP and become more anti-apoptotic. It has been shown that red light and IR in the range from 580 nm to 830 nm can protect cells from oxidative damage. The effect of the phototherapy light is believed to be an increase in ATP production and an increase in ATP stores within the cells. This gives the cells the energy reserves to resist the damage from oxidative stress. The photodissociation of nitric oxide causes the re-establishment of ATP production in cells, particularly when the cells have been under mild inflammatory attack in the case of diseases like keratoconus. The anti-apoptotic effects of red light have been observed in human dermal cells and have been demonstrated to reduce pain from injury, speed up the healing process and reduce cellular damage created in the wound healing response. There are currently FDA approved devices for phototherapy in applications involving the dermis, and phototherapy has been demonstrated to rescue retinal cells from oxidative damage done by chemical ingestion of methanol. Since the object of the chemical reaction of UVA and riboflavin is to produce reactive oxygen species, red light phototherapy is well suited to reduce oxidative cellular injury and potential cellular loss caused during the photochemical treatment.

As noted above, the action spectrum for the phototherapeutic light extends from 580 nm to 830 nm. The red light phototherapeutic dose is biphasic in that either too much or too little light does not achieve the desired result. The literature reports that about 4.5-5.0 J/cm² over a 5-minute period is a good fluence for dermal cells. Due to the light shielding of the dermal cells by the stratum corneum and melanin it is determined that the optimal biphasic dose for exposed ocular cells is 3.0-3.5 J/cm² over a 10 minute period. Since the interaction of red light with mitochondria is common to all cells, the effect is common to all cell types. Those cells whose function requires more energy have more mitochondria and these cells are particularly well protected. This implies an irradiance of about 5 mw/cm² for the 10-minute period. The time of the irradiation is important because the red light only photodissociates nitric oxide (a competitive inhibitor of oxygen) during certain cycles of the electron transport chain. The time for the electron transport chain to process electrons is about 10 minutes, so the time of phototherapy is extended to catch all of the various electron transport chains contained in the mitochondria during the appropriate part of the cycle. The treatment time may be appropriately controlled by the physician using these considerations. In one embodiment, red light phototherapy is delivered in a wavelength range of 580 nm to 830 nm in a dose in the range of 2.0 J./cm² to 6.0 J./cm² for a three to seven minute period.

As discussed above, the treatment system also includes monitoring system 40 for the photoluminescence emitted from the riboflavin interaction with UVA/blue light, using optical collection device 158 as illustrated in FIG. 10. This photoluminescence consists of fluorescence from the riboflavin photonic emission from the S1 to S0 state and phosphorescence emitted from the triplet riboflavin state. These photoluminescent emissions can give a measure of riboflavin concentration in the cornea, a relative measure of the depth of penetration of the riboflavin into the stroma, a relative measure of the lateral homogenity of the riboflavin and a relative measure of the oxygen utilization and triplet state formation. The reaction of riboflavin and UVA/blue radiation involves two electronically excited states of riboflavin. When ground state S0 (unexcited riboflavin) absorbs UVA/blue light it transitions into an excited state called the S1 state. From the excited S1 state, the molecule can lose its energy by two mechanisms. The first mechanism is the relaxation back to the ground state by emitting a photon of light in a process called fluorescence. The peak fluorescence of riboflavin is about 525 nm. The average quantum yield for riboflavin in aqueous solutions is about 0.3, meaning that the ratio of photons emitted/photons absorbed is about 0.3. The second mechanism for relaxation from the S1 state is called the formation of triplet riboflavin and this is accomplished by a mechanism called intersystem crossing. The triplet state of riboflavin is the only excited state that can impart energy to molecular oxygen and create singlet oxygen for cross-linking. From this triplet state the riboflavin molecule can react and give up the excess energy to oxygen or water, or it can phosphoresce to the ground state. The phosphorescence of triplet riboflavin occurs at around 605 nm. Since phosphorescence is a direct measure of the active species that creates singlet oxygen, optical collection device 158 and optical monitoring device 40 of FIG. 13 are configured to monitor both the fluorescent and phosphorescent signals.

Optical collection device 158 of FIG. 10 comprises light collection lens 83 and bifurcated light guide 70 which receives the light collected by lens 83. This bifurcated light guide has one single end that splits into two output ends 70A, 70B. The lens 83 is directed to the center of the cornea and receives the photoluminescent emissions from the cornea and focuses these emissions to the proximal or receiving end 185 of the single-ended portion 70 of the bifurcated light guide. The light guide is designed to provide 50% of the proximal input light into each of the two distal light guide portions 70A, 70B. The distal portions of the light guides are routed to the optical monitoring module 40 shown in layout view on FIG. 13. The optical heads or ends 185 on each of the light guides 70, 71 of the treatment devices 150 and 151 receive the photoluminescent emissions from the irradiated corneas of the patient's left and right eyes, and the emission light is transmitted by light guides 71 and 70, respectively, into left eye guide portions 71A, 71B and right eye guide portions 70A, 70B. The photoluminescent emission from each eye includes both fluorescence and phosphorescence due to different types of riboflavin interactions, as discussed in detail below. The emission light is directed onto filters 42 and 43 for separating fluorescence emission from phosphorescence emissions for each eye, as illustrated in FIG. 13. Filter 42 is a narrowband bandpass filter with a center wavelength of 525 nm-535 nm to capture the peak of the fluorescence emission from the riboflavin. Filter 43 is a narrowband bandpass filter centered at 600 nm-605 nm to capture the peak of the phosphorescence of the triplet riboflavin. By splitting the emissions collected from each eye being treated, both the phosphorescence and fluorescence for each eye may be monitored. The filtered emission light from each light guide is directed onto a respective sensor 41, which may comprise a PIN silicon photodiode 41 which may incorporate an integral preamplifier or thermoelectric cooling, and the output voltage of the photodiode is transmitted to high impedance amplifier 45 for conversion of the photonic energy into voltage. Alternatively items 41 and 45 can be purchased as an integral unit from commercial sources such as Thorlabs and are capable of detection of signals as small as a few femptowatts (10-15 watts).

As illustrated in FIG. 4A, the output signals from the four amplifiers shown in FIG. 13 can be routed to the microprocessor 17 in the illumination source 10 for A to D conversion, signal processing and display. It is known from the Beer Lambert law that for a given wavelength and excitation energy the fluorescent intensity of a sample is linearly proportional to the concentration of the fluorophore, in this case riboflavin. The relationship I=kcPO shows that the intensity of the fluorescence (I) is a linear function of the concentration (c) of the riboflavin and the intensity of the applied excitation light (P0). This is illustrated by test data using the treatment system described above and riboflavin solution of known concentration.

In one example, an optical projection head in the system illustrated and described above using UVA at 365 nm and a 9 mm projection spot was directed upon four samples of diluted riboflavin to detect the fluorescence intensity. A hypotonic 0.1% riboflavin solution was diluted 2:1, 3:1 and 4:1 and fluorescent intensity was obtained from a ½″ collecting optic and 600 micron single fiber with the collecting optic placed as shown in FIG. 10. The fluorescent light was routed by the fiberoptic to a femptowatt silicon photodiode system and filtered by a 532 nm center wavelength narrowband filter (Edmund Optics NT-097). The recorded fluorescent intensity readings were displayed as raw millivolts on a digital voltmeter. A plot of the data obtained in this test is shown in FIG. 20. Background readings were not subtracted from the raw data readings. It is concluded that the fluorescent intensity has a linear correlation with the riboflavin concentration and the fluorescence signal output from monitoring module 40 may thus be used to measure corneal riboflavin concentration.

In one embodiment, the intensity of the riboflavin photoluminescence from the cornea may be measured by a filtered photodiode in the monitoring system or module 40 and compared to a standard sample of a known concentration. A reference calibration standard with known concentration may be supplied with the system, or the user can use a supplied cuvette and the 0.1% riboflavin used for the treatment may be used as the calibration standard. When the standard of known concentration is placed at 75 mm from the optical projection lens, a background radiation reading is taken to act as a zero point. Then the standard is irradiated with a known intensity of UVA/blue light and fluorescent intensity is converted into a voltage reading at the optical monitoring module and can be stored by the microprocessor. The difference between the zero point reading and the calibration standard reading gives the conversion factor for calculating the riboflavin concentration. For example if the background reading of the room light is 250 millivolts and the standard fluorescent reading at 3.0 mw/cm² of UVA is 2250 millivolts, we know that the concentration of 0.1% riboflavin represents the difference in voltage of 2000 millivolts when the UVA intensity is set to 3.0 mw/cm². Then the optical head is positioned over the riboflavin soaked cornea and illuminated with 3.0 mw/cm² of UVA. If the fluorescence reading is 1750 millivolts, then the difference of the corneal riboflavin fluorescence and the background reading is 1750-250 or 1500 millivolts. A comparison is made of the reading of the cornea to the reading of the standard to determine the riboflavin concentration in the cornea. In this case the corneal concentration of riboflavin is 1500/2000×0.1% or 0.075% riboflavin.

The monitoring of the fluorescence provides an additional benefit in that it reflects a change in the UVA intensity, which may signal a potential problem with the light source or light transmission system. The illumination system shown in FIG. 4 has been demonstrated to be stable within 1% over a 30 minute period if the system is warmed up for 10 minutes prior to use. An alarm can be built into the system that would sound if the fluorescence changed more than 10% from the calibration intensity. On actuation of the alarm, the operator can first check the UVA/blue light calibration to see if the output value has changed. If the output value remains as set, then the drop in fluorescence is due to riboflavin photobleaching and the user could then add additional riboflavin to the cornea.

An alternative construction for the riboflavin concentration measurement uses colorimetry rather than photoluminescence for riboflavin determination. The colorimetric method uses a red, green, and blue (RGB) sensor in lieu of photodiode 41, and filters 42 and 43 are removed from the optical monitoring module 40. These RGB color detection sensors are widely used in industrial and commercial applications to match colors and automated monitoring of product defects by color matching. Such sensors can easily fit into the end of a pen and are commercially available from many companies such as Texas Advanced Optoelectronic Solutions (TAOS), Omron and other companies. Riboflavin is a highly yellow colored dye because it absorbs light in the blue spectral region, and the RGB sensors can measure the amount of blue light reflected from the cornea in relation to reflected red and green light. By analysis of the ratios of blue light to red light the amount of riboflavin in the cornea can be ascertained. Since the light reflected from the cornea may vary as a function of the ambient room light, color of the iris and the vascular state of the retina, a baseline calibration of the cornea is taken before riboflavin is added. Then a sterilizable transparent yellow filter of known riboflavin absorption is placed over the cornea to establish a known riboflavin calibration point. As an example the transparent yellow filter might represent the blue light absorbed by 400 micromolar riboflavin solution. As riboflavin is added to the cornea, the ratio of detected blue light to red light decreases as a function of the amount of additional riboflavin. The change in the ratio of blue to red reflected light detected by the sensor following riboflavin administration is the result of absorption of blue light by the riboflavin in the cornea. Thus, comparison of this ratio with the calibration ratio detected using the calibration filter determines the riboflavin concentration. Monitoring of the changes of fluorescence during the irradiation procedure can be done by the blue and green components of the RGB sensor to provide information on the rate of change of riboflavin fluorescence (green) and riboflavin phosphorescence (red) to accomplish the oxygen monitoring functions. If a colorimetric sensor is used then the signals from the optical monitoring module are processed by the microprocessor using the ratios of blue/red light from the cornea compared to the calibration reading.

In one method of using the optical treatment system described above to stimulate corneal cross-linking, a photosensitizer which is typically riboflavin is added to the eye in advance of treatment and one or both treatment units are then positioned at the predetermined working distance from the cornea using the positioning apparatus. The treatment wavelength (UVA or blue) is selected along with other variable parameters such as continuous or discontinuous irradiation, signal intensity, pulse time for discontinuous radiation, beam shape, and size. After all variables are set and a sufficient amount of riboflavin has diffused into the cornea, treatment is commenced. Additional topical solutions may be applied to the eye at periodic intervals during treatment, for example, riboflavin and other solutions such as solutions for enhancing cross-linking, for example by providing additional oxygen, as described in more detail below. Addition of more riboflavin or other solutions may be based on the riboflavin concentration determined from the output of photoluminescence monitoring module 40.

Once the concentration of riboflavin in the cornea is determined using the monitoring and processing steps described above, the operator can use this information to determine if more riboflavin needs to be infused prior to the light treatment, or the operator can select an irradiation intensity that increases or maximizes the cross-linking while still protecting the endothelium. The calculation of the optimal intensity can be done by hand, by nomogram or by the microprocessor 17, but the process is the same. The data required for the calculation is the thickness of the cornea and the concentration of the riboflavin. It is standard for the physician to measure the thickness of the cornea prior to the procedure by pachymetry, and the concentration has been determined by the fluorescent intensity compared to the calibration standard. The goal in protecting the endothelium is to prevent a toxic dose of UVA being delivered to the endothelial cells. The literature reports this toxic dose as 0.35 mw/cm² of UVA at the endothelium over a 30-minute irradiation, or a total fluence of 0.63 J/cm² of UVA on the endothelium when riboflavin is present. A calculation can be made of the maximum intensity of the applied radiation that accumulates 0.63 J/cm² of UVA at the endothelium. For a cornea that is 500 microns thick with a detected concentration of riboflavin of 0.075%, the Beer Lambert law can be used to calculate absorbance and transmission of UVA. The 0.075% percentage solution is first converted to molarity so the known molar extinction coefficients can be used. Multiplying the % solution by 10 and dividing by the formula weight gives the molarity of the percent solution. In this instance the concentration is approximately 1.6 millimolars. Applying the known quantities to Beers Law, A=Ebc gives the absorbance of the 1.6 millimolar solution at 500 microns as A=0.83 and the transmission percentage at 500 micron is 15%. Since 15% of the incident radiation is reaching the endothelium it is calculated that a maximum of 4.2 J/cm² may be applied on the incident surface of the cornea. However, additional adjustment for radiation absorbed by non-riboflavin chromophores in the stroma also needs to be considered. It is also known that only 67% of the incident light in a cornea that is not infused with riboflavin reaches the endothelium due to absorption by other chromophores, so the incident energy may be increased by ⅓ and still be at the toxic threshold, i.e. a maximum of 5.5 J/cm² can be placed incident to the cornea. This means the operator may select 3 mw/cm² for 30 minutes or 6 mw/cm² for 15 minutes and the endothelium receives 0.63 J/cm². Naturally a safety margin can be applied by the physician and the irradiance or irradiation time may be cut by a safety factor that the physician believes appropriate. As stated earlier the calculations above would not be done by the physician but by nomogram or by the microprocessor, and the physician only needs to enter the thickness of the cornea into the microprocessor, using input device 98 which may be a suitable keypad input on the control panel or associated with a separate microprocessor.

The Beer Lambert law assumes that the riboflavin is homogeneously dispersed in the solutions being measured. This assumption may not be correct but can be tested by using two different wavelengths of light. In the above example the 3.0 mw/cm² UVA irradiation value provided a 1500 millivolt change in fluorescence indicating a 0.075% average riboflavin concentration in the 500 micron cornea. Since the fluorescence is stoichiometric, a blue 405 nm wavelength with different penetration may be used to see if the blue light confirms a homogenous dispersion of the riboflavin. If the cornea is irradiated with 2.7 mw/cm² of blue light, the number of blue photons striking the riboflavin is the same number of photons as UVA at 3.0 mw/cm². The blue light penetrates deeper due to its lower molar extinction coefficient and the calculation of the transmission and absorption by the Beer Lambert equations show A=0.63 and the transmission at 500 microns is 23.7%. It can be calculated that the fluorescence of UVA corresponds to 85% absorption in the 500 micron cornea but only 76.3% of the blue light is absorbed in the cornea. Given the proportionally less fluorescence of the blue light as compared to UVA, a reading of 76.3/85.0×1500=1200 millivolts is expected for the blue light fluorescence if the riboflavin is homogenous in the stroma. A blue light fluorescence around 1200 millivolt confirms a homogenous distribution of riboflavin. If the riboflavin is mostly at the surface, the deeper penetrating blue light does not have as many interactions with riboflavin and the result is a low reading. For example, a reading of 800 instead of 1200 indicates that the riboflavin is concentrated in the anterior levels of the stroma where it is strongly fluoresced by the UVA but only partially fluoresced by the deeper penetrating blue light. This information provides an indication of the depth of riboflavin penetration in the stroma, which is useful for UVA 370 mm light treatment purposes, and also gives the user the option to switch from UVA to blue light for the excitation. The lower concentration of riboflavin in the posterior stroma has better opportunity to cross link more with blue light given the lower molar extinction coefficient and deeper penetration.

The phosphorescence of the riboflavin triplet state can be used to monitor the efficiency of the reaction particularly with relation to singlet oxygen formation. Each eye is monitored for both fluorescence and phosphorescence using optical collection devices 158 and photoluminescence monitoring unit 40, as described above. It is expected that, during the course of the irradiation, the riboflavin photo-oxidizes and degrades to a form that does not fluoresce or create triplet molecules. We would expect under ideal conditions that the phosphorescence would degrade at the same rate. However, it is known that the presence of oxygen is required for phosphorescence of riboflavin to occur in solutions, and that oxygen also quenches the phosphorescence of the riboflavin. The quenching of the phosphorescence by oxygen can be measured by the reduction in the phosphorescence signal. Since some degradation in the triplet phosphorescence signal is expected as a result of riboflavin degradation, the optimal index for monitoring the oxygen quenching of triplet riboflavin is the ratio of the phosphorescence to the fluorescence. The phosphorescence signal can be compared to the fluorescence signal during calibration and expressed as a ratio. For example, this might be 30:100 for the initial reading. As the reaction proceeds over time, the ratio drops as the phosphorescence signal drops which reflects quenching of triplet riboflavin by molecular oxygen. This drop in the ratio may be used as a proxy measure of the singlet oxygen production. As the ratio of the phosphorescent/fluorescent signal drops, this means the efficiency of singlet oxygen production is dropping, and this ratio levels off at some point. This may be used as a signal for the operator to reoxygenate the cornea by discontinuous illumination. The shutter 12 in FIG. 4 provides discontinuous illumination and the operation of the shutter cycle can be directed by the operator by the softkey inputs on the control panel shown in FIG. 6.

Oxygen is consumed during cross-linking and needs to be replenished. This replenishment is done through the anterior corneal surface. When excitation energy is applied to the surface of the cornea, the oxygen that is reentering the cornea is consumed at a rate that exceeds the reoxygenation diffusion rate and the cornea remains hypoxic, particularly in the posterior portions, under continuous wave conditions. It is noted that blue light excitation gives the user an option for increased reoxygenation of the posterior stroma. Blue light is less absorbed in the anterior cornea and accordingly the oxygen consumption rate is lowered. This allows more of the replenishment oxygen to reach the posterior stromal region.

It is known that the triplet riboflavin molecules created during photochemical therapy can either form singlet oxygen created in a Type II reaction or hydrogen peroxide by a Type I reaction. In the presence of physiological amounts of oxygen of 20 mm Hg partial pressure the Type II singlet oxygen reaction predominates. Under conditions of subnormal oxygen availability (less than 5 mm Hg of O2), the Type I hydrogen peroxide reaction predominates. It has been demonstrated that the stromal region is hypoxic under the current protocol of continuous 3.0 mw/cm2 UVA and 0.1% riboflavin cornea. The available oxygen content of the stroma is consumed almost immediately as demonstrated by the following calculation. Given the volume occupied by a 500-micron thick cornea and the reported literature value of 35 micromolar oxygen in the stroma, the total amount of oxygen in the cornea is about 1.4×10-9 moles. The quantum yield of singlet oxygen from riboflavin irradiation is 0.52 meaning that approximately 2 photons of absorbed energy consumes 1 unit of molecular oxygen. This means that only 2.8×10-9 moles of photons are required to consume all of the available stromal oxygen. Using the relationship E=hv the amount of energy to deplete all of the cornea oxygen is less than 1 mj of UVA light. It is reported that 97% of the UV is absorbed in the cornea under the current cross-linking protocols. This means that the total available oxygen in the stroma is consumed in about ⅓ second after irradiation commences. Even if the riboflavin concentration were only 1/10 of the literature reports, all of the oxygen would be consumed in about 3 seconds.

In one embodiment of a corneal treatment method using the system described above, an additional topical solution may be applied separately to the eye or in a mixture with the riboflavin solution in order increase the oxygen available to the cornea for cross-linking purposes and to simultaneously reduce the amount of toxic hydrogen peroxide produced during hypoxia. This application may take place before treatment with UVA or blue light commences, during treatment, or both. Because oxygen is replenished to the cornea from the overlying tear film, and the tear film is only 5 microns thick on average ( 1/100th of the stromal thickness), it would require the oxygen content of 100 individual fully oxygenated tear films to replenish all the oxygen in a 500-micron thick stroma. Replenishment of the oxygen in a tear film either requires blinking or re-equilibration of the tear film from the atmosphere. Blinking is usually difficult during photochemical cross-linking because the eyelids are usually held open by a clamp during the procedure. The literature reports that an unoxygenated tear film can require up to 60 seconds to reestablish its level of dissolved oxygen from the atmosphere.

One potential solution to reoxygenating the tear film is to use drops of pre-oxygenated aqueous ophthalmic solutions with high partial pressures of oxygen. Such solutions are disclosed in US Patent Application Publication No. 20080057023. However, this teaching only partially addresses the high oxygen needs in photochemical cross-linking due to the low solubility of oxygen in aqueous saline solutions. An improved means of reoxygenating the stroma is disclosed herein and is composed of lipid or oil-based fluids that are pre-oxygenated at high oxygen partial pressures (350-760 mm Hg) for topical application to the cornea. One example of a suitable fluid is the synthetic oil perfluorodecalin, which can carry 100 times more oxygen per volume as an aqueous saline solution. Perfluorodecalin is also UVA/blue light transparent, biologically inert and FDA approved for human use. One 5-micron tear film of perfluorodecalin pre-oxygenated at 300 mm Hg of oxygen could completely reoxygenate the entire stroma and still have a remaining partial pressure of oxygen equal to the partial pressure of oxygen in the atmosphere. The high partial pressure provides the driving force to transfer the oxygen into the stroma. Other oil based fluids which may be used for this purpose include silicone oils that have 50-75 times the oxygen carrying capacity of saline, and olive oil or mineral oil with about 25 times the oxygen carrying capacity of saline. These oils can be used in pure form or as part of emulsions. In pure form, oils such as mineral oil float on top of the epithelial mucin layer if the epithelium is intact, or on top of Bowmans' membrane if the epithelium has been removed. Advantageously, these hydrophobic oil solutions do not migrate into the aqueous stroma. An additional advantage of the oxygenated oil fluids is that they all allow passage of CO₂ from the corneal stroma and do not absorb ions from the cornea that could upset the osmotic balance or thickness of the stroma. A known affect of applying oils onto the cornea is that oils can disrupt the barrier function of the epithelium. However, this barrier disruption is desirable in photochemical cross-linking, since it facilitates the infusion of riboflavin into the stroma.

The oxygenation of the oils can be accomplished by bubbling oxygen through the oils or by applying high-pressure oxygen mixtures to the oils. For example, bubbling 50% oxygen 50% nitrogen solution through the oil results in a an oil with a partial pressure of about 380 mm Hg oxygen.

The stroma remains hypoxic once irradiation has commenced due to the relatively slow oxygen uptake rate of the cornea. The maximum corneal oxygen uptake (COU) rate for an open eye is about 10 microliters-O₂ cm²/hour or about 1.2×10-10 moles-O₂ cm²/second. Energy being applied at 3.0 mw/cm2 consumes oxygen in the photochemical process at a rate 30 times greater than the maximum reported COU. This leads to a continuous hypoxic state of the stroma during irradiation, and creates the conditions for high Type II hydrogen peroxide production in the stroma. It is known that UV or visible light interacts with hydrogen peroxide to form hydroxyl ions which are the most toxic of all reactive oxygen species to living cells. Even though the oxygen consumption rate of UVA/blue light and riboflavin far exceeds the replenishment rate of oxygen into the stroma, the low oxygen content shifts the quantum yield of singlet oxygen to very low numbers (0.02 or less) and some of the migrating oxygen diffuses into the anterior stroma giving the limited anterior cross-linking observed in the current procedure.

In another embodiment, additional oxygen for cross-linking may be obtained from the hydrogen peroxide that inevitably results from the Type II riboflavin reactions during hypoxia. In one embodiment, this hydrogen peroxide is converted into water and oxygen by introducing a hydrogen peroxide reducing agent into the stroma. Suitable reducing agents for this purpose are the enzyme catalase or iodide anions. Most of the molecular oxygen obtained from the hydrogen peroxide does not originate from dissolved atmospheric oxygen, but is new oxygen extracted by from water in the stroma. This new photochemically and catalytically produced molecular oxygen provides a new source of oxygen for singlet oxygen production. The same process simultaneously serves to reduce the toxic levels of hydrogen peroxide and hydroxyl ions. It has been demonstrated that riboflavin Type II reactions can create tissue hydrogen peroxide in concentrations ranging from 50 to 500 micromolar. It is also known that as little as 1 micromolar hydrogen peroxide can stop the proliferation of living cells. The addition of catalase and/or iodide ion to the cornea or to solutions used during photochemical cross-linking has the simultaneous effect of reducing toxicity and improving singlet oxygen production. Catalase is a large molecule with a molecular weight of about 250,000 and its diffusion into the stroma is very slow. Since catalase is slow to diffuse into the cornea, the iodide ion may be a better stromal catalyst for hydrogen peroxide decomposition. The iodide ion is very small, and may penetrate past the epithelium into the stroma very quickly since the epithelium is either removed or the barrier function is reduced to allow riboflavin infusion. Once the iodide is past the epithelial barrier it could be evenly distributed throughout the stroma within minutes. Iodide anion and catalase both act as catalysts to decompose hydrogen peroxide into water and molecular O₂, but iodide ion can act as a catalyst for hydrogen peroxide decomposition under conditions that could inactivate catalase. For example, in the presence of nitric oxide, catalase is rendered inactive but iodide ion continues to decompose hydrogen peroxide. In one embodiment, sodium iodide is used as the iodide ion for photochemical cross-linking, although other forms of iodide ion are acceptable. Iodate ion (IO3-) administered with a reducing agent such as bisulfite ion (HSO3-) may be used for introducing the iodide ion. It is known that high levels of iodate ion can destroy the integrity of the retinal pigment epithelium, so caution must be exercised when iodate ion is used to form the iodide ion inside the eye.

In one embodiment, an ophthalmic or artificial tear solution may contain a concentration of iodide such as sodium iodide in ranges from 0.1 to 130 mmol. The concentration range for catalase varies by the activity of the type of catalase used. Catalase has different activity depending on its source (microbial, bovine blood or liver, human blood, etc.). In one embodiment, sufficient catalase to decompose 1 millimolar hydrogen peroxide within 1 minute may be added to the riboflavin solution. Since the catalysts remain unchanged during the peroxide decomposition, the concentration of the catalyst only affects the overall rate of reaction for the hydrogen peroxide decomposition. The decomposition of hydrogen peroxide is exothermic and too much iodide could result in unwanted heat if peroxide concentrations are high, and too little iodide may result in increased hydroxide ion production from the UV/blue light since the hydrogen peroxide is in solution longer. Potassium iodide is a less preferred form of iodide salt during photochemical cross-linking since the potassium ion can adversely affect riboflavin triplet formation. Other hydrogen peroxide decomposition catalysts are available but have drawbacks. For example FeCl₃ decomposes hydrogen peroxide but produces large amounts of hydroxyl ion by the Fenton reaction. Catalysts like MnO₂ or PbO form particulates in the cornea that are difficult for the ocular circulatory system to remove. Other peroxidase enzymes can be used in lieu of catalase, but these have the general drawback of decomposing the hydrogen peroxide by reactions with other substrates and molecular oxygen is not the main byproduct of the reaction. The addition of these non-catalase peroxidases to riboflavin solutions or to the cornea comprise an improvement over current compositions and procedures, but are limited to detoxifying the hydrogen peroxide without creating new molecular oxygen. It is known that glutathione peroxidases are not as well suited as catalase to decompose hydrogen peroxide in high levels.

The addition of iodide ion to the stroma during cross-linking quenches or reduces riboflavin fluorescence and reduces single oxygen quantum yield. This quenching effect is considered advantageous in that it reduces the rate at which oxygen re-entering the stroma is consumed and allows for greater oxygen penetration and deeper stromal cross-linking.

The inclusion of catalase and/or sodium iodide as part of the riboflavin formulations applied to the eye for corneal cross-linking treatment or other purposes provides a significant improvement to the current packaged riboflavin solution. Current commercial aqueous-based riboflavin solutions are often provided in clear glass or plastic containers that allow ambient light to activate the riboflavin and create singlet oxygen and hydrogen peroxide in the bottle. The singlet oxygen production in the bottle has two deleterious effects. First, the singlet oxygen degrades the riboflavin content and reduces the effective riboflavin concentration. Second, the creation of singlet oxygen depletes the dissolved oxygen content of the solution. When this is placed on the cornea, the anoxic fluid serves to remove oxygen from the stroma rather than replenish oxygen to the stroma. The hydrogen peroxide created in the solution bottle is also placed onto the cornea and has toxic consequences to the cornea. All of these drawbacks to existing riboflavin preparations are eliminated by the addition of catalase and/or sodium iodide to the riboflavin preparation.

A further advantage of including catalase and/or iodide ion in the riboflavin preparation is that the resulting mixture can be lyophilized for greater stability during storage and transport, and then it may be reconstituted with a dilute hydrogen peroxide mixture. The advantage of this method is that the added hydrogen peroxide is converted into molecular oxygen in the reconstituted riboflavin solution, and high partial pressures of oxygen can be achieved. For example using a solution of 400-micromolar hydrogen peroxide to reconstitute lyophilized riboflavin mixture containing catalase or iodide anion provides enough oxygen to saturate the solution to 300 mm of Hg of oxygen. This provides double the capacity of available oxygen in the solution compared to an atmospherically exposed riboflavin solution. The riboflavin and peroxide solutions should be placed in actinic glass or UV and visible light protected gas impervious plastic containers.

Another method of reoxygenating the stroma is by applying drops of a highly oxygenated aqueous based solution containing iodide ion. The aqueous solutions do not match the oxygen carrying capacity of oils but they can be used to replace the iodide ion in the stroma and the iodide ion generates new oxygen in the stroma from hydrogen peroxide. Iodide does not tend to dwell for long periods in the stroma and disperses into the anterior chamber and into neighboring tissue from the stroma. This dispersion is advantageous since it can also alleviate potential damage from peroxide that has migrated out of the stroma.

The use of topical solutions for increasing oxygen concentration of the eye either by introducing a hydrogen peroxide reducing agent into the stroma or by adding pre-oxygenated solutions to the eye, or both, also increases the amount of oxygen available for cross-linking. The discontinuous illumination option described above also provides for reoxygenation and the prevention of peroxide build up. The option of blue light excitation also offers the user an additional option for greater oxygen penetration.

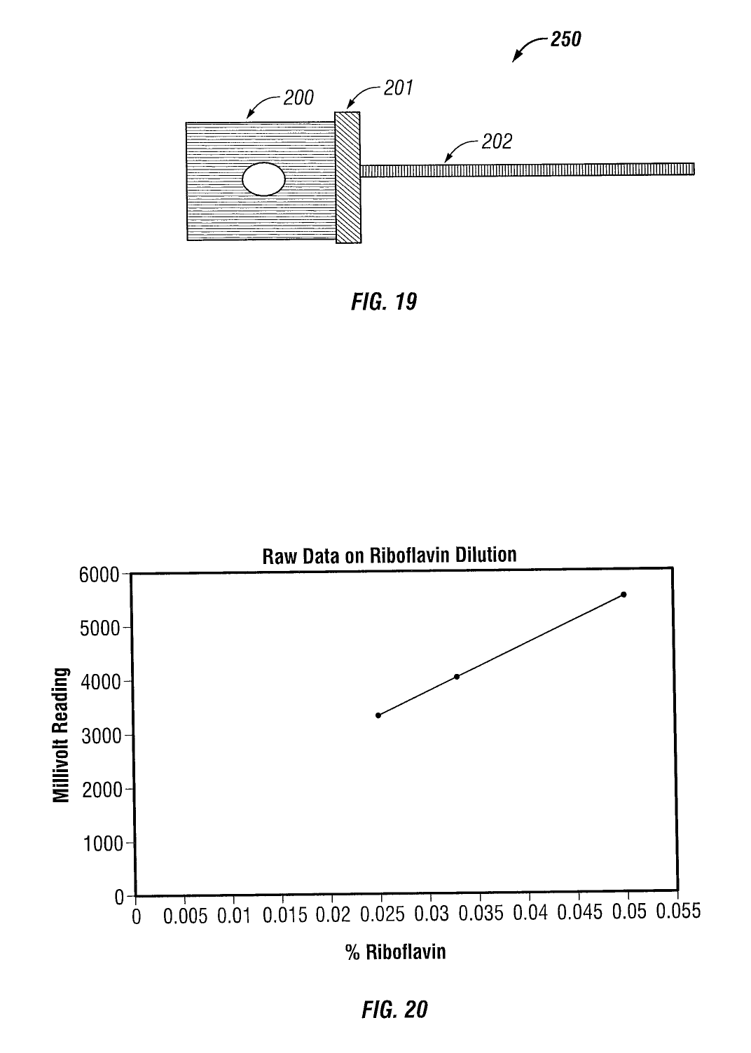

It has been noted by slit lamp examination that the central or inferior stromal regions appear to infuse with more riboflavin than the peripheral edges or inferior region of the cornea. It is desirable to have an indication of the homogenity of the lateral dispersion of the riboflavin. FIG. 19 illustrates one embodiment of a hand held dodging fixture 250 that can be used to provide a relative measure of the lateral dispersion of the riboflavin. Item 200 is a UV transparent/visible blocking glass with a 3 mm hole drilled into the center. Commercial glasses like Schott UG 11 are suitable for use with UVA and Schott BG 4 for is suitable for use with blue light at 405 nm. The excitation energy can reach the cornea, although somewhat diminished and the only emitted light to reach the sensing optic on the optical head would be through the hole in the center of the glass. Item 201 is a plastic holder for the filter and handle 202.

The dodging fixture can be held over the central cornea and the resultant fluorescence reading from optical monitoring module 40 then reflects the emissions only from the central hole in the fixture. The fixture then can be moved laterally to different areas of the cornea to obtain readings relative to the central cornea. The dodging tool, therefore, can provide a relative quantitative measure of lateral dispersion. For optimal signal to noise ratio a black cloth or mask of opaque material placed over the patient's surgical field with a hole cut into the cloth for the eyes reduces the background radiation and provide less background radiation and a better relative measure. If the readings show the central cornea has loaded with more riboflavin than the peripheral areas then the physician may wait for a longer period for the riboflavin to disperse, or may take other action to promote dispersion, such as placing a warm cloth over the closed eye for a few minutes or using other dispersion techniques to speed up the lateral riboflavin dispersion.

The systems and methods described above allow for bilateral or monocular photochemical cross-linking of corneal collagen employing selectable UVA/blue light as the excitation source and riboflavin as the photosensitizer. One embodiment of the system has an illumination source with multi-spectral capability, light guides for delivery of light to bilateral optical heads for projection onto the corneal surface of both eyes simultaneously, selectable radiation patterns to accommodate individual corneal architecture, and red light phototherapy to limit apoptosis and accelerate healing time. In the foregoing embodiments, the light source is a Hg or Xe short arc lamp, which is more stable than the LED sources used in prior art systems. The light source is connected to the treatment head or heads via liquid light guides, which produces improved homogeneity in the light beam. The image projection optics is designed to produce a relatively large working distance between the treatment head and the eye, which is at least 50% greater than the working distance in prior art corneal treatment systems. This provides better visualization for the surgeon as well as better access for diffusion augmentation techniques and the like. In some embodiments, provision may be made for an adjustable working distance. The spot size and shape on the eye can also be modified easily by selecting from a kit of different apertures or masks.

Aiming beams allow accurate alignment of the optical head at a predetermined spacing from the patient cornea. A microprocessor-controlled rotary solenoid mechanical shutter provides the option of discontinuous illumination for tissue reoxygenation. In one embodiment, devices and methods are also included for the in situ determination of the riboflavin content and approximate riboflavin depth of penetration in the cornea.

In some or all of the foregoing embodiments, a highly oxygenated topical solution may be placed on the cornea for stromal reoxygenation during cross-linking treatment, such as a solution containing iodide ion or a lipid or oil-based fluid that is pre-oxygenated at a high oxygen partial pressure. In another alternative, a hydrogen peroxide reducing agent or solution is applied to the eye which can convert hydrogen peroxide produced in the stroma during irradiation of the eye into oxygen and water. Suitable reducing agents for application to the eye for this purpose are topical solutions containing iodide ion or the enzyme catalase, as described in detail above. These agents may be added to any standard riboflavin solution or as a separate solution applied to the cornea during photochemical treatment.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

We claim:

1. A corneal treatment system, comprising:
an illumination source;
an optical assembly associated with the illumination source and configured to direct light along a predetermined light path from the illumination source;
the optical assembly including at least one elongate light guide defining part of the light path and a wavelength control device configured to direct radiation in at least one predetermined wavelength band along the light path and to prevent transmission of radiation outside said at least one wavelength band along the light path; and
at least one optical treatment head mounted in the light path to receive radiation in said one wavelength band, the optical treatment head including an optical projection system configured to direct a treatment light beam onto a patient's eye at a predetermined working distance from the optical head, whereby the optical treatment head can be positioned at a spacing from the patient's eye sufficient to allow access to the eye by a physician during treatment.

2. The system of claim 1, wherein the working distance is at least two inches.

3. The system of claim 2, wherein the working distance is approximately three inches.

4. The system of claim 1, wherein the optical treatment head includes an aperture which is configured to control at least the size of the light beam at the predetermined working distance from the optical head.

5. The system of claim 4, wherein the aperture is adjustable to vary the size of the light beam projected onto a patient's eye at the predetermined working distance from the patient's head.

6. The system of claim 5, further comprising a mask holder in the optical treatment head and a plurality of masks configured for removable mounting in the mask holder, each mask having an opening configured to define the aperture, the masks having openings of different sizes to vary the size of the aperture.

7. The system of claim 6, wherein the masks include masks having openings of different shapes.

8. The system of claim 1, wherein the optical projection system comprises a lens having a predetermined focal length.

9. The system of claim 8, wherein the working distance is greater than the focal length of the lens, wherein the beam size is magnified by a predetermined amount at the working distance from the optical treatment head.

10. The system of claim 1, wherein at least part of the light path comprises a flexible, UV transmissive liquid light guide extending up to the optical head.

11. The system of claim 1, wherein the illumination source is a multi-spectral light source.

12. The system of claim 11, wherein the illumination source is an LED light engine.

13. The system of claim 11, wherein the illumination source is a short arc lamp which emits UVA light and light of other wavelengths.

14. The system of claim 13, wherein the optical assembly further comprises a beam isolator configured to direct light in at least a predetermined UVA wavelength range of approximately 330 to 380 nm to the wavelength control device and to prevent transmission of light outside the predetermined wavelength range along the light path from the beam isolator.

15. The system of claim 14, wherein the beam isolator is configured to direct light in a predetermined wavelength range including both UVA and blue light to the wavelength control device.

16. The system of claim 15, wherein the predetermined wavelength range is 340 nm to 470 nm.

17. The system of claim 14, wherein beam isolator comprises at least one UVA/blue light reflective dichroic mirror which reflects UVA and blue light along the light path and transmits light outside the UVA/blue light range out of the light path.

18. The system of claim 1, wherein the illumination source is a short arc lamp selected from the group consisting of short arc mercury lamps, short arc mercury halide lamps, and short arc xenon lamps.

19. The system of claim 1, wherein the wavelength control device is configured for selective transmission of light in at least two different predetermined wavelength bands along the optical path to the optical treatment head.

20. The system of claim 19, wherein the wavelength control device comprises at least first and second filters and a controller configured for selectively positioning the first or second filter in the light path, the first filter comprising a UVA filter and the second filter comprising a blue light filter, whereby UVA light or blue light can be selectively focused on a patient's eye for treatment purposes.

21. The system of claim 20, wherein the first filter comprises a UVA filter having approximately 10 nm bandwidth at 365 nm and the second filter comprises a blue light filter having approximately 10 nm bandwidth at 405 nm.

22. The system of claim 1, further comprising a support stand, an adjustable mounting assembly on the support stand, the optical treatment head supported on the mounting assembly, wherein the mounting assembly is configured for X and Y directional adjustment of the position of the optical treatment head relative to a patient's eye.

23. The system of claim 22, wherein the mounting assembly comprises an articulated arm having a first end mounted for vertical sliding adjustment on the support stand and a second end supporting the optical treatment head.

24. The system of claim 22, wherein the mounting assembly includes a swivel joint configured for adjustment of the angle of the light beam directed from the optical treatment head to a patient's eye.

25. The system of claim 22, further comprising a second optical treatment head, the optical treatment heads comprising right and left treatment heads configured for directing separate right and left treatment light beams onto the right and left eyes of a patient, the mounting assembly having right and left end portions associated with the right and left optical treatment heads, respectively.

26. The system of claim 25, wherein the mounting assembly comprises an articulated arm assembly having a first portion slidably associated with the support stand, the right and left end portions being pivotally connected to the first portion.

27. The system of claim 26, wherein one of the end portions is articulated.

28. The system of claim 26, wherein the mounting assembly further comprises first and second swivel joints between the right and left end portions and the right and left optical treatment heads, respectively.

29. The system of claim 28, further comprising locking devices configured for releasably locking each optical treatment head at a selected X, Y and Z adjusted position relative to a respective eye of a patient.

30. The system of claim 1, comprising right and left optical treatment heads each including projection optics and configured to project respective right and left light beams onto the respective right and left eyes of a patient at a predetermined working distance from the optical head, the light guide including separate right and left guide portions connected to the right and left optical treatment heads, respectively.

31. The system of claim 30, wherein at least the right and left guide portions of the light guide are flexible light guides, and each optical treatment head is adjustably mounted for at least x and y direction adjustment of the position of the optical treatment head relative to a patient's eye.

32. The device of claim 30, further comprising an intensity adjustment module configured to adjust the intensity or irradiance of the light beam emitted by each optical treatment head independently.

33. The system of claim 30, further comprising an adjustable mounting assembly configured for adjusting the separation between the right and left optical treatment heads and the distance of each optical treatment head from a patient's eye.

34. The system of claim 33, wherein the adjustable mounting assembly includes independent swivel joints configured for adjusting the angle of each optical head relative to a patient's eye.

35. The system of claim 1, further comprising an adjustment mechanism configured for varying the distance of the optical treatment head from a patient's eye and a positioning apparatus associated with the optical treatment head and configured to indicate to an operator when the optical treatment head is at the working distance from the patient's eye, whereby the operator can position the optical treatment head at the predetermined working distance from the patient's eye.

36. The system of claim 35, wherein the positioning apparatus comprises first and second aiming devices located on opposite sides of the optical treatment head and configured to emit visible light aiming beams at an angle to the optical axis of the treatment head which cross over one another at the predetermined working distance from the treatment head.

37. The system of claim 36, wherein the first and second aiming devices comprise laser diodes each mounted at a predetermined angle to the optical treatment head such that angled light beams emitted by the diodes cross over at the predetermined working distance from the optical treatment head, whereby the optical treatment head is at the working distance from a patient's eye when a spot formed at the cross over point of the angled light beams is positioned on a patient's eye.

38. The system of claim 37, wherein each aiming device further comprises an attenuation filter positioned in the path of light emitted by laser diode to reduce the power of the angled light beams.

39. The system of claim 38, wherein each aiming device comprises a red light emitting diode (LED).

40. The system of claim 39, wherein each aiming device further comprises a light shaping diffuser movably associated with the aiming device and configured to be moved between a retracted position and an advanced position in the path of light emitted by the LED to shape the red light beam into a circular beam of predetermined size at the working distance for selectively applying red light phototherapy to a patient's eye.

41. The system of claim 37, wherein the positioning apparatus comprises an aiming device mounted on one side the optical treatment head and configured to direct a second light beam at an angle to the optical axis of the optical treatment head which crosses the optical axis at the selected working distance from the optical treatment head, a sensor device mounted on the opposite side of the optical treatment head from the aiming device and oriented at a predetermined angle to receive any light from the second light beam which is reflected from a patient's eye at the predetermined working distance, and a monitor for monitoring the amount of light received by the sensor device, whereby the optical treatment device is at the selected working distance from a patient's eye when the amount of light received is at a maximum.

42. The system of claim 41, wherein the aiming device comprises a light emitting diode (LED) and the sensor device comprises a photodiode.

43. The system of claim 1, further comprising at least one multi-component treatment unit, the optical treatment head comprising part of the treatment unit.

44. The system of claim 43, wherein the treatment unit further comprises a support base on which the optical treatment head is mounted.

45. The system of claim 43, wherein the treatment unit further comprises a red light phototherapy device configured to provide red light phototherapy to the eye of a patient undergoing treatment.

46. The system of claim 43, wherein the treatment unit further comprises a positioning device configured for determining when the optical treatment head is at the predetermined working distance from a patient's eye.

47. The system of claim 43, wherein the treatment unit further comprises an optical collection device configured to collect photoluminescent emissions from the cornea of a patient undergoing treatment.

48. The system of claim 47, further comprising a monitoring module configured to measure the intensity of riboflavin induced photoluminescent emissions from the cornea, and a light guide connecting the output of the optical collection device to the monitoring module.

49. The system of claim 48, wherein the monitoring module comprises a first bandpass filter connected to the output of the optical collection device, the first bandpass filter having a center wavelength corresponding to the peak of fluorescence emission of riboflavin, and a first sensor which receives the output of the first bandpass filter output to produce a first output signal dependent on the detected fluorescence emission from the eye of a patient being treated.

50. The system of claim 49, wherein the monitoring module further comprises a second bandpass filter connected to the output of the optical collection device in parallel with the first bandpass filter, the second bandpass filter having a center wavelength corresponding to the peak of phosphorescence of triplet riboflavin, a second sensor which receives the output of the second bandpass filter to produce a second output signal dependent on the intensity of detected phosphorescence from the eye of a patient being treated.

51. The system of claim 50, further comprising a processor which receives the first and second output signals and which is configured to process the first output signal and produce an output signal which varies in response to variations in riboflavin concentration in the cornea of a patient.

52. The system of claim 1, further comprising a shutter selectively movable into the light path from the illumination source to the treatment head to cut off the light beam emitted by the treatment head, and a controller configured to control movement of the shutter into and out of the light path for discontinuous illumination of a patient's eye.

53. The system of claim 52, wherein the controller has an input for operator selection of the on-off time period for discontinuous illumination.

54. The system of claim 1, further comprising a red light treatment device configured to provide red light phototherapy to the eye of a patient undergoing UVA treatment using the optical treatment head.

55. The system of claim 54, further comprising a shut off device for shutting off the light beam emitted from the optical treatment head during red light phototherapy.

56. The system of claim 1, further comprising a monitoring device configured to monitor the concentration of riboflavin in the cornea of a patient undergoing treatment and a display unit having an output corresponding to the monitored riboflavin concentration.

57. The system of claim 56, further comprising an intensity control device for varying the intensity of the light beam emitted by the optical treatment head, whereby an operator can vary the therapeutic light intensity dependent on the monitored riboflavin concentration.

58. A method of producing a light beam for use in phototherapy treatment of the eye, comprising:
directing radiation from an illumination source along an optical path from the illumination source;
isolating radiation in a predetermined wavelength range from the radiation emitted from the illumination source, and directing the isolated radiation along the optical path;
filtering the isolated radiation to eliminate all radiation outside at least one predetermined wavelength band and directing the filtered radiation along the optical path;
mounting a first optical treatment head in the optical path to receive the isolated, filtered light; and
directing a light beam from the optical treatment head and focusing the beam to produce a spot of predetermined size and shape at a predetermined working distance from the optical treatment head, whereby the optical treatment head can be positioned at a spacing from the patient's eye sufficient to allow access to the eye by a physician during treatment.

59. The method of claim 58, further comprising splitting the radiation along separate first and second optical paths, mounting the first optical treatment head in the first optical path and mounting a second, identical optical treatment head in the second optical path, whereby the first and second optical treatment heads comprise right and left eye treatment heads, respectively, and both eyes of a patient can be treated simultaneously.

60. The method of claim 59, further comprising adjusting the separation between the optical treatment heads based on a patient's eye spacing, adjusting the distance of the respective treatment heads from the eyes until the treatment heads are at the predetermined working distance from the eyes, and securing the optical treatment heads at the adjusted positions.

61. The method of claim 60, further comprising adjusting the angle of each optical treatment head independently dependent on an operator selected orientation of a light beam to be applied to the respective eye of a patient, and securing the optical treatment head at the selected angle during treatment.

62. The method of claim 59, further comprising adjusting the intensity of light applied to each eye independently.

63. The method of claim 58, further comprising positioning the optical treatment head at the predetermined working distance from a patient's eye prior to treatment, the step of positioning the optical treatment head comprising directing at least a first aiming light beam at an angle to the optical axis of the optical treatment head such that the light beam intersects the optical axis at a point located at said predetermined working distance, and adjusting the position of the optical treatment head until the intersection point of the aiming light beam coincides with the surface of a patient's eye.

64. The method of claim 63, further comprising directing a second aiming light beam at an opposite angle from the first light beam towards the optical axis whereby the first and second aiming light beams intersect at said point, such that a spot of light is formed on a patient's eye when the optical treatment head is at said predetermined working distance from the patient's eye.

65. The method of claim 63, further comprising monitoring for reflection of said aiming light beam from a surface located at said intersection point, whereby a maximum amount of reflected light is detected when said optical treatment head is at the predetermined working distance from a patient's eye located at said intersection point, and the optical treatment head is secured in position when a maximum amount of reflected light is detected.

66. The method of claim 58, further comprising turning the treatment light beam on and off at predetermined intervals to provide discontinuous illumination of an eye.

67. The method of claim 58, wherein said at least one wavelength band is a predetermined UVA wavelength band.

68. The method of claim 58, wherein said at least one wavelength band is a predetermined blue light wavelength band.

69. The method of claim 58, wherein the illumination source is a multi-spectral light source.

70. The method of claim 69, further comprising selectively filtering the isolated light beam in two different wavelength bands to provide treatment light beams of different wavelengths, the first wavelength band comprising a predetermined UVA wavelength band and the second wavelength band comprising a predetermined blue light wavelength band.

71. The method of claim 67, further comprising applying red light phototherapy to a patient's eye during UVA light therapy.

72. The method of claim 67, further comprising applying red light phototherapy to a patient's eye before UVA light therapy.

73. The method of claim 67, further comprising applying red light phototherapy to a patient's eye after UVA light therapy.

74. The method of claim 67, further comprising shutting off the UVA light beam at periodic intervals to provide a discontinuous UVA light treatment cycle.

75. The method of claim 74, further comprising applying red light phototherapy to a patient's eye during periods of the discontinuous UVA light treatment cycle when the UVA light beam is shut off.

76. The method of claim 58, further comprising introducing a topical riboflavin solution for photochemical collagen cross-linking into a patient's eye.

77. The method of claim 76, further comprising monitoring the level of photoluminescent emissions from a patient's eye during treatment and determining approximate riboflavin concentration in the cornea based on the level of photoluminescent emissions.

78. The method of claim 77, further comprising introducing more riboflavin solution into the eye if the riboflavin concentration falls below a predetermined level.

79. The method of claim 77, further comprising varying the intensity of the UVA light beam in response to variations in the determined riboflavin concentration.

80. The method of claim 76, wherein the riboflavin topical solution comprises a mixture of riboflavin or a riboflavin derivative and iodide ion.

81. The method of claim 76, wherein the riboflavin topical solution comprises a mixture of riboflavin or a riboflavin derivative and catalase.

82. The method of claim 58, further comprising introducing an oxygenated topical solution onto the cornea of a patient's eye prior to or during photochemical treatment using the light beam, the solution comprising at least 155 mm Hg partial pressure of oxygen.

83. The method of claim 82, wherein the topical solution comprises 20% to 100% of an oxygenated oil or oil-based liquid.

84. The method of claim 76, further comprising introducing iodide ion into the stroma.

85. The method of claim 84, wherein the riboflavin topical solution includes iodide ion.

86. The method of claim 76, wherein the riboflavin topical solution includes sodium iodide.

87. The method of claim 58, further comprising controlling the size of an aperture in the optical treatment head, whereby the size of the spot of light at a patient's eye can be varied depending on the eye condition being treated and the size of a patient's eye.

88. The method of claim 58, further comprising varying the shape of the aperture, the step of varying the shape of the aperture comprising selecting from a plurality of different aperture shapes comprising at least round, elliptical and annular.

89. The method of claim 58, further comprising introducing a hydrogen peroxide reducing agent into the stroma of a patient's eye during treatment.

90. The method of claim 89, wherein the hydrogen peroxide reducing agent is selected from the group consisting of iodide ion, catalase, and naturally extracted peroxidase enzyme.

91. A system for photochemical cross-linking of corneal collagen, comprising:
an illumination source;
an optical system associated with the illumination source and configured to provide an output of radiation in a predetermined ocular treatment wavelength range;
an optical treatment head connected to the output, the optical treatment head including an optical projection system configured to direct a treatment light beam onto a patient's eye;
a mounting stand; and
an adjustable mounting assembly which adjustably mounts the optical treatment head on the stand and is configured for X, Y and Z direction adjustment of the position and orientation of the optical treatment head relative to a patient's eye.

92. A system for photochemical cross-linking of corneal collagen, comprising:
an illumination source;
an optical system associated with the illumination source and configured to provide an output of radiation in a predetermined ocular treatment wavelength range; and
an optical treatment unit comprising an optical treatment head connected to the output and including an optical projection system configured to direct a treatment light beam onto a patient's eye, and a red light emitting device for selectively directing a red light phototherapy beam onto a patient's eye.

93. A system for photochemical cross-linking of corneal collagen, comprising:
an illumination source;
an optical system associated with the illumination source and configured to provide a first output of radiation in a predetermined ocular treatment wavelength range;
an optical treatment unit comprising an optical treatment head connected to the first output and including an optical projection system configured to direct a treatment light beam onto a patient's eye, and light collection device configured to collect riboflavin induced photoluminescent emissions from the cornea of an eye during photochemical treatment of the cornea and to produce a second output which varies with intensity of the collected photoluminescent emissions.

94. The system of claim 93, further comprising a monitoring module connected to the light collection device and configured to process the output to produce an output signal proportional to the concentration of riboflavin in the cornea, and an output display unit which displays the output signal, whereby an indication of current estimated riboflavin concentration in the cornea is provided to an operator of the system

95. A method of photochemical cross-linking of corneal collagen, comprising:
applying a riboflavin solution containing a hydrogen peroxide reducing agent to the stroma; and
irradiating at least a predetermined area of the cornea with radiation in a predetermined UVA or blue light wavelength band at a selected intensity for a selected treatment period.

96. The method of claim 95, wherein the hydrogen peroxide reducing agent is iodide ion.

97. The method of claim 96, wherein the iodide ion in the solution is between 0.1 and 100 millimolar.

98. The method of claim 95, wherein the peroxide reducing agent is catalase.

99. The method of claim 98, wherein the amount of catalase in the solution is sufficient to decompose 1 millimolar of hydrogen peroxide within one minute.

100. The method of claim 98, wherein the peroxide reducing agent is a naturally extracted peroxidase enzyme.

101. The method of claim 95, wherein the radiation is UVA radiation in a predetermined wavelength band of approximately 355 nm to 375 nm.

102. The method of claim 95, wherein the radiation is blue light radiation in a predetermined wavelength band of approximately 395 nm to 415 nm.

103. A method of photochemical treatment of the cornea to produce photochemical collagen cross-linking, comprising:
applying a riboflavin solution to the stroma;
irradiating at least a predetermined area of the cornea with UVA radiation in a predetermined wavelength band at a selected intensity for a selected treatment period; and
applying a highly oxygenated topical solution on the cornea prior to or during irradiation of the stroma, the solution comprising at least 155 mm. Hg. Partial pressure of oxygen.

104. The method of claim 103, wherein the topical solution contains between 20% to 100% of an oxygenated oil.

105. The method of claim 103, wherein the topical solution is an aqueous based solution or emulsion containing iodide ion.

106. The method of claim 103, wherein the concentration of iodide ion in the solution is between 0.1 and 100 millimolar.

107. An ocular treatment composition comprising a solution containing iodide ion.

108. The composition of claim 107, wherein the iodide ion in the solution is in the range of 0.1. to 100 millimolar.

109. The composition of claim 107, wherein the iodide ion is sodium iodide.

110. The composition of claim 107, wherein the solution contains riboflavin.

111. An ocular treatment composition comprising riboflavin or a riboflavin derivative for photochemical cross-linking and catalase.

112. The composition of claim 111, wherein the catalase level in the solution is sufficient to decompose one millimolar of hydrogen peroxide in one minute.

113. An ophthalmologic phototherapy treatment system, comprising:
an illumination source;
a photosensitizing agent adapted for delivery to the cornea; and
a phototherapy system configured to deliver light in the range of 580 nm to 830 nm to the cornea.

114. A method of ophthalmologic phototherapy treatment, comprising:
applying a photosensitizing agent to the cornea; and
irradiating the cornea with light in the range of 580 nm to 830 nm in a dose of approximately 2.0 J/cm2 to 6.0 J/cm2 for a selected time period.

115. The method of claim 114, wherein the selected time period is three to seven minutes.

116. A photochemical corneal treatment system, comprising:
an illumination source;
a riboflavin photosensitizing agent adapted for delivery to the cornea;
a detector configured to receive riboflavin induced photoluminescent emissions from the cornea and to produce an output signal which varies with intensity of the received photoluminescent emissions; and
a monitoring unit connected to the output of the detector and configured to process the output signal to determine riboflavin concentration in the cornea.

117. A method of detecting riboflavin concentration in a cornea during photochemical treatment of the cornea, comprising:
applying a riboflavin solution to the cornea;
irradiating the cornea with radiation in a predetermined photochemical treatment range;
detecting photoluminescent emissions from the cornea during treatment; and
determining approximate riboflavin concentration in the cornea based on the intensity of the detected photoluminescent emissions.

118. A method of detecting riboflavin concentration in a cornea during photochemical treatment of the cornea, comprising:
placing a yellow filter corresponding to a known riboflavin concentration over the cornea;
illuminating the cornea;
detecting a first ratio of blue light reflected from the cornea through the yellow filter relative to light of at least one second color reflected from the cornea, the first ratio comprising a calibration point;
removing the yellow filter and applying a riboflavin solution to the cornea;
detecting a second ratio of blue light reflected from the cornea relative to reflected light of the at least one second color; and
comparing the first and second ratios to determine approximate riboflavin concentration in the cornea.
